(19) United States
(12) Patent Application Publication
(10) Pub. No.: US 2017/0224310 A1
(43) Pub. Date: Aug. 10, 2017

(54) ULTRASONIC DIAGNOSTIC APPARATUS AND ULTRASONIC SIGNAL PROCESSING METHOD

(71) Applicant: Konica Minolta, Inc., Tokyo (JP)

(72) Inventor: MASARU FUSE, Osaka (JP)

(21) Appl. No.: 15/384,935

(22) Filed: Dec. 20, 2016

(30) Foreign Application Priority Data
Feb. 4, 2016 (JP) 2016-019655

(51) Int. Cl. A61B 8/08; A61B 8/00

(52) U.S. Cl. CPC A61B 8/5207; A61B 8/54; A61B 8/461; A61B 8/4483

(57) ABSTRACT

An ultrasonic diagnostic apparatus that transmits/receives an ultrasonic wave to/from a subject using an ultrasonic probe and generates an image includes: a transmission unit that converts a pulsed transmission signal including a fundamental wave component into a transmission ultrasonic wave and transmits the transmission ultrasonic wave to the inside of the subject; a receiving unit that generates a reception signal based on a reflected ultrasonic wave from the subject; a separation unit that separates the reception signal into first and second components; a phase control unit that generates a third component by controlling a phase of the second component such that a time at which amplitude is maximized is the same between the first and second components; a combining unit that combines the first and third components to generate a composite reception signal; and an image generation unit that generates an image based on the composite reception signal.

The entire disclosure of Japanese Patent Application No. 2016-019655 filed on Feb. 4, 2016 including description, claims, drawings, and abstract are incorporated herein by reference in its entirety.

### BACKGROUND OF THE INVENTION

Field of the Invention

The present invention relates to an ultrasonic diagnostic apparatus and an ultrasonic signal processing method, and in particular to a method of transmitting and receiving ultrasonic waves.

Description of the Related Art

An ultrasonic diagnostic apparatus is a medical imaging apparatus that acquires information of the inside of the body using an ultrasonic pulse reflection method and displays the information as a tomographic image. By taking advantage of low cost, no risk of exposure to radiation, and excellent real-time performance compared with other modalities using X-rays, radiation, and the like, the use area of the ultrasonic diagnostic apparatus is expanding.

Various studies for improving the image quality in the ultrasonic diagnostic apparatus have been made. For example, a technique called tissue harmonic imaging (THI) is used. The THI is a technique of extracting and imaging nonlinear components generated when ultrasonic waves propagate through the body tissue, specifically, harmonic components. In addition to being used for imaging of the body tissue itself, the THI can also be used to generate a contrast image in combination with an ultrasonic contrast agent for generating strong harmonic components. Since each harmonic component has a higher frequency than the fundamental wave component, the harmonic component is less susceptible to the influence of multiple reflection, low-frequency noise, and the like. In addition, since the amount of unnecessary side lobe components is small, it is possible to obtain a signal with a high S/N ratio. In addition, for example, as disclosed in JP 2004-298620 A or JP 2010-42048 A, by using a plurality of harmonics having different orders or by using a sum frequency or a difference frequency corresponding to two fundamental waves having different frequencies, signal quality has been improved due to an increase in the band of a signal.

As another advantage of using a component having a higher frequency than the fundamental wave component, it is possible to improve the distance resolution by reducing the time length of the pulse (hereinafter, abbreviated as a pulse length) of the ultrasonic wave. However, since harmonic components are generated when the fundamental wave component propagates, there is almost no change in the pulse length between the fundamental wave component and the harmonic components. Therefore, in a method of simply using harmonic components such as that disclosed in JP 2004-298620 A, it is not possible to improve the distance resolution since the time length of the pulse is not different from that of the fundamental wave.

As a method of reducing the pulse length, as disclosed in JP 2010-42048 A, there is a method in which a signal (so-called “chirp signal”) whose frequency changes (sweeps) with time is transmitted and received and pulse compression using correlation processing is used. However, in order to sweep the ultrasonic frequency, analog processing is required. For this reason, there is a problem that the circuit is complicated and the cost is increased.

### SUMMARY OF THE INVENTION

The invention has been made in order to solve the aforementioned problem, and it is an object of the invention to provide an ultrasonic diagnostic apparatus and an ultrasonic signal processing method which can be realized by simple processing and by which it is possible to achieve both an increase in the band of a signal and an improvement in distance resolution.

To achieve the abovementioned object, according to an aspect, an ultrasonic diagnostic apparatus that transmits and receives an ultrasonic wave to and from a subject using an ultrasonic probe and generates an image based on a reflected ultrasonic wave, reflecting one aspect of the present invention comprises: a transmission unit that converts a pulsed transmission signal including a fundamental wave component into a transmission ultrasonic wave using the ultrasonic probe and transmits the transmission ultrasonic wave to the inside of the subject; a receiving unit that generates a reception signal based on a reflected ultrasonic wave from the subject that has been received by the ultrasonic probe; a separation unit that separates the reception signal into a first component including one or more frequency components and a second component different from the first component; a phase control unit that generates a third component by controlling a phase of the second component such that a time at which amplitude is maximized is the same between the first and second components; a combining unit that combines the first and third components to generate a composite reception signal; and an image generation unit that generates an image based on the composite reception signal.

### BRIEF DESCRIPTION OF THE DRAWINGS

The above and other objects, advantages and features of the present invention will become more fully understood from the detailed description given hereinbelow and the appended drawings which are given by way of illustration only, and thus are not intended as a definition of the limits of the present invention, and wherein:

FIG. 1 is a block diagram of an ultrasonic diagnostic apparatus according to a first embodiment;

FIG. 2 is a flowchart showing the operation of the ultrasonic diagnostic apparatus according to the first embodiment;

FIG. 3 is a flowchart showing the operation of a transmission and reception event according to the first embodiment;

FIG. 4A shows an example of the waveform of a transmission pulse according to the first embodiment;

FIG. 4B shows an example of the waveform of a transmission pulse according to the first embodiment;

FIG. 5A shows an example of the waveform of a composite reception signal according to the first embodiment;

FIG. 5B shows an example of the waveform of a composite reception signal according to the first embodiment;

FIG. 6 is a block diagram of an ultrasonic diagnostic apparatus according to a first modification example;

FIG. 7 is a flowchart showing the operation of the ultrasonic diagnostic apparatus according to the first modification example;

FIG. 8A shows an example of the band of a transmission signal pulse according to a second modification example;

FIG. 8B shows an example of the band of a digital reception signal according to the second modification example;

FIG. 9A is a schematic diagram showing components to be processed by a separation unit, a phase control unit, and a combining unit according to the first embodiment;

FIGS. 9B to 9D are schematic diagrams showing components to be processed by a separation unit, a phase control unit, and a combining unit according to a third modification example;

FIG. 10 is a block diagram of an ultrasonic diagnostic apparatus according to a second embodiment;

FIG. 11 is a flowchart showing the operation of a transmission and reception event according to the second embodiment;

FIG. 12 shows examples of a reference signal according to second and third embodiments;

FIG. 13 is a block diagram of an ultrasonic diagnostic apparatus according to the third embodiment;

FIG. 14 is a flowchart showing the operation of a transmission and reception event according to the third embodiment;

FIG. 15 is a block diagram of an ultrasonic diagnostic apparatus 6 according to a fourth embodiment;

FIG. 16 is a flowchart showing the operation of a transmission and reception event according to the fourth embodiment;

FIG. 17 is a schematic diagram of estimation correction according to the fourth embodiment;

FIG. 18 is a block diagram of an ultrasonic diagnostic apparatus according to a fifth embodiment;

FIG. 19 is a flowchart showing the operation of a transmission and reception event according to the fifth embodiment;

FIG. 20A is a schematic diagram showing an example of the combination ratio between a fundamental wave component and a nonlinear component in a combining unit;

FIG. 20B is a schematic diagram showing the relationship between the depth in a subject and the generation level of a nonlinear component;

FIG. 20C is a schematic diagram showing the relationship between the attenuation rate of each of a fundamental wave component and a nonlinear component and the depth in a subject; and

FIG. 20D is a schematic diagram showing the relationship between the signal level of each of a fundamental wave component and a nonlinear component and the depth in a subject.

### DESCRIPTION OF THE PREFERRED EMBODIMENTS

Hereinafter, an embodiment of the present invention will be described with reference to the drawings. However, the scope of the invention is not limited to the illustrated examples.

<<Circumstances that Led to Embodiments for Carrying Out the Invention>>

The inventors have performed various studies to achieve both an improvement in signal quality based on an increase in the band of a signal due to the THI and an improvement in distance resolution due to using a high-frequency signal.

In the THI, harmonic components are extracted and imaged. Since each harmonic component has a higher frequency than the fundamental wave component, the harmonic component is less susceptible to the influence of multiple reflection, low-frequency noise, and the like. In addition, since the amount of unnecessary side lobe components is small, it is possible to obtain a signal with a high S/N ratio. In addition, since the harmonic component has a higher frequency than the fundamental wave component, the transmission beam is easily narrowed. Accordingly, there is a characteristic that the azimuth resolution is high.

On the other hand, the inventors have found a problem that it is not possible to improve the distance resolution just by using the harmonic components. This is because the distance resolution depends on the pulse length of the ultrasonic wave. In general, the distance resolution improves as the frequency of the ultrasonic pulse increases. This is because the pulse length becomes shorter as the frequency becomes higher if the wave number is the same. However, although the frequency of the harmonic component is higher than the frequency of the corresponding fundamental wave component, the pulse length itself of the harmonic component is the same as the pulse length of the fundamental wave component. For this reason, the distance resolution in the THI is not improved more than the distance resolution in the case of imaging the fundamental wave component. Then, the inventors have studied techniques for shortening the pulse length using the harmonic component.

As a known technique for shortening the pulse length, for example, as disclosed in JP 2010-42048 A, a pulse compression technique using correlation processing can be mentioned. In the method disclosed in JP 2010-42048 A, harmonic components are separated for each order of the harmonic, pulse compression of a second harmonic, a third harmonic, a fourth harmonic, and a fifth harmonic is performed, and the results are combined. In this technique, however, a chirp signal is used as a transmission pulse. In order to generate a chirp signal, analog processing for the frequency sweep is required. For this reason, the method disclosed in JP 2010-42048 A causes the complication of circuits and a cost increase.

Then, the inventors have studied a method of shortening the pulse length by making the peak steep by combining a plurality of different frequency components in a reception signal. For example, in the method disclosed in JP 2004-298620 A, two fundamental waves having different frequencies are used as transmission waves, and the phases of fundamental waves in a transmission ultrasonic wave are controlled. Accordingly, in the reflected ultrasonic wave, a second harmonic corresponding to one of the fundamental waves and a difference frequency component or a sum frequency component between the fundamental waves are combined so as to strengthen each other. In these techniques, however, such combination cannot be performed, for example, so that the fundamental wave component and the second harmonic component strengthen each other or the third harmonic and the component of the sum frequency strengthen each other. This is because, in the phase control of the transmission ultrasonic wave such as that performed in JP 2004-298620 A, the phase of the component of the difference frequency or the sum frequency can be made to match the phase of each component of the even harmonics group (a second harmonic, a fourth harmonic, and the like), but the phase of each component of the even harmonics group cannot be made to match the phase of the fundamental wave component and each component of the odd harmonics group (a third harmonic, a fifth harmonic, and the like). That is, it is not possible to strengthen the fundamental wave component and the second harmonic component each other. Therefore, the inventors have obtained the idea of shortening the pulse length by making the peak steep by strengthening the respective components of the reception signal by controlling the phase of each component of the reception signal instead of the phase of the transmission signal.

Hereinafter, an ultrasonic diagnostic apparatus and an ultrasonic signal processing method according to an embodiment will be described in detail with reference to the diagrams.

### First Embodiment

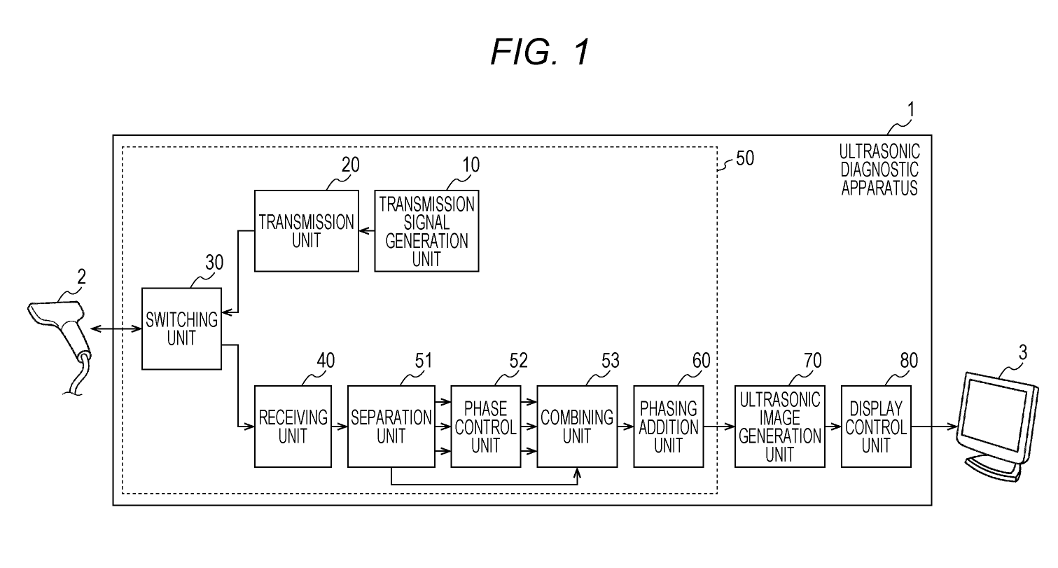

FIG. 1 shows a block diagram of an ultrasonic diagnostic apparatus 1 according to a first embodiment. The ultrasonic diagnostic apparatus 1 includes a transmission signal generation unit 10, a transmission unit 20, a switching unit 30, a receiving unit 40, a separation unit 51, a phase control unit 52, a combining unit 53, a phasing addition unit 60, an ultrasonic image generation unit 70, and a display control unit 80. In addition, the transmission signal generation unit 10, the transmission unit 20, the switching unit 30, the receiving unit 40, the separation unit 51, the phase control unit 52, the combining unit 53, and the phasing addition unit 60 form an ultrasonic signal processing circuit 50. In addition, an ultrasonic probe 2 is configured so as to be connectable to the switching unit 30, and a display unit 3 is configured so as to be connectable to the display control unit 80. FIG. 1 shows a state in which the ultrasonic probe 2 and the display unit 3 are connected to the ultrasonic diagnostic apparatus 1.

The ultrasonic probe 2 has a plurality of transducers (not shown) arranged in a one-dimensional direction, for example. Each transducer is formed of, for example, PZT (lead zirconate titanate). The ultrasonic probe 2 converts an electrical signal (hereinafter, referred to as an “element driving signal”) generated in the transmission unit 20 into an ultrasonic wave. The ultrasonic probe 2 transmits an ultrasonic beam, which is formed by a plurality of ultrasonic waves emitted from a plurality of transducers, to a measurement target in a subject in a state in which the transducer-side outer surface of the ultrasonic probe 2 is in contact with a surface such as the skin surface of the subject. Then, the ultrasonic probe 2 receives a plurality of reflected ultrasonic waves from the measurement target, converts each of the reflected ultrasonic waves into an electrical signal (hereinafter, referred to as an “element reception signal”) using the plurality of transducers, and supplies the element reception signal to the switching unit 30.

The transmission signal generation unit 10 is a circuit for generating a transmission signal for generating an element driving signal. The transmission signal generation unit 10 generates a pulse signal having a frequency in a predetermined frequency band that is a fundamental wave component, for example, a pulse signal having a center frequency of 4 MHz. Here, the pulse signal is a sine wave (cosine wave) in principle, and is not a continuous wave but a signal having a finite length of about one to several periods. In addition, the transmission signal generation unit 10 may further generate a pulse signal, which corresponds to the harmonic component and has a frequency of integral multiples of the fundamental wave component, combine the pulse signal with a pulse signal of the fundamental wave component, and output the resulting signal.

The transmission unit 20 is a circuit for performing the focusing or steering of the ultrasonic beam based on the transmission signal by setting the delay time for each transducer. Specifically, for the transmission timing of the ultrasonic beam, delay time is set for each transducer. Then, by delaying the transmission signal generated by the transmission signal generation unit 10 by the delay time, an element driving signal is generated for each transducer. The element driving signal is, for example, a pulsed electrical signal of different timing for each transducer element, which is generated such that transmission ultrasonic waves, which are transmitted from the transducer elements that form the ultrasonic probe 2, become focus waves that reach a transmission focus point at the same time. Alternatively, the element driving signal may be, for example, a pulsed electrical signal which is generated such that the transmission ultrasonic waves, which are transmitted from the transducer elements that form the ultrasonic probe 2, become plane waves traveling in a specific direction and which is obtained by setting the same timing for each transducer element or by shifting the operation timing stepwise at a fixed pitch from one end to the other end of the transducer column.

The switching unit 30 selects a transducer of the ultrasonic probe 2 to be driven by the element driving signal, and connects the selected transducer and the transmission unit 20 to each other. In addition, the switching unit 30 selects a transducer of the ultrasonic probe 2 to generate an element reception signal, and connects the selected transducer and the receiving unit 40 to each other.

The receiving unit 40 converts each element reception signal based on the reflected ultrasonic wave into a digital reception signal by performing amplification and then A/D conversion of the element reception signal.

The separation unit 51 is a circuit for separating the digital reception signal for each frequency band and outputting a fundamental wave component to the combining unit 53 and outputting nonlinear components to the phase control unit 52. Here, the nonlinear components refer to components other than the fundamental wave component, specifically, harmonic components. Alternatively, the separation unit 51 may output a component the timing of the peak of which is the same as that of the fundamental wave component, among the nonlinear components, to the combining unit 53 together with the fundamental wave. Alternatively, for example, the separation unit 51 may output the fundamental wave component and a component the timing of the peak of which is the same as that of the fundamental wave component, among the nonlinear components, to the phase control unit 52, and output the remaining components of the nonlinear components to the combining unit 53. Separation for each frequency band can be performed using a band pass filter, for example. Alternatively, the separation for each frequency band may be performed using a band pass filter after using a phase inversion method to be described later.

The phase control unit 52 is a circuit for controlling one or both of the phase of the fundamental wave component and the phase of the nonlinear component so that the timing of the peak of the nonlinear component output from the separation unit 51 is the same as that of the fundamental wave component, that is, a phase indicating the peak is the same as that of the fundamental wave component. The details thereof will be described later. As used herein, “the same” is intended to cover substantially the same in a scope capable of providing an intended effect.

The combining unit 53 is a circuit for generating a composite reception signal by combining the fundamental wave component output from the separation unit 51 and the nonlinear component output from the phase control unit 52 in a predetermined combination ratio so that the timings match each other. The combining unit 53 amplifies one or both of the fundamental wave component and the nonlinear component according to the combination ratio, and adds the fundamental wave component and the nonlinear component after the amplification.

The phasing addition unit 60 is a circuit for performing phasing addition for the composite reception signal to generate an acoustic line signal. In a case where the transmission ultrasonic wave is a focus wave, the acoustic line signal based on the reflected ultrasonic wave is generated for regions obtained by dividing a region of interest, which is a part of a region through which the transmission ultrasonic wave has passed and which includes the transmission focus point and the vicinity thereof, in the element column direction. Accordingly, in a case where the transmission ultrasonic wave is a focus wave, in order to obtain the acoustic line signal of the entire region of interest, transmission of the transmission ultrasonic wave and reception of the reflected ultrasonic wave are repeatedly performed while moving the transmission focus point in the element column direction. On the other hand, in a case where the transmission ultrasonic wave is a plane wave, the transmission ultrasonic wave is transmitted so as to spread over the entire region of interest, and the acoustic line signal of the entire region of interest is generated based on the reflected ultrasonic wave.

The ultrasonic image generation unit 70 is a circuit for generating a B-mode image signal by performing envelope detection, brightness conversion using logarithmic compression, and coordinate transformation to the orthogonal coordinate system for a plurality of acoustic line signals required when constructing one tomographic image.

The display control unit 80 is a circuit for displaying the B-mode image signal generated by the ultrasonic image generation unit 70, as an image, on the display unit 3.

The display unit 3 is an image display device connected to the display control unit 80. For example, the display unit 3 is a liquid crystal display or an organic EL display.

The transmission signal generation unit 10, the transmission unit 20, the switching unit 30, the receiving unit 40, the separation unit 51, the phase control unit 52, the combining unit 53, the phasing addition unit 60, the ultrasonic image generation unit 70, and the display control unit 80 are realized by hardware, such as a field programmable gate array (FPGA) and an application specific integrated circuit (ASIC), for example. In addition, two or more of these may be configured as a single element. For example, the ultrasonic signal processing circuit 50 may be configured as a single FPGA. In addition, some or all of these may be realized by a single FPGA or ASIC. In addition, these may be realized separately or with two or more thereof as one using a memory, a programmable device such as a central processing unit (CPU) and a graphic processing unit (GPU), and software.

<Operation>

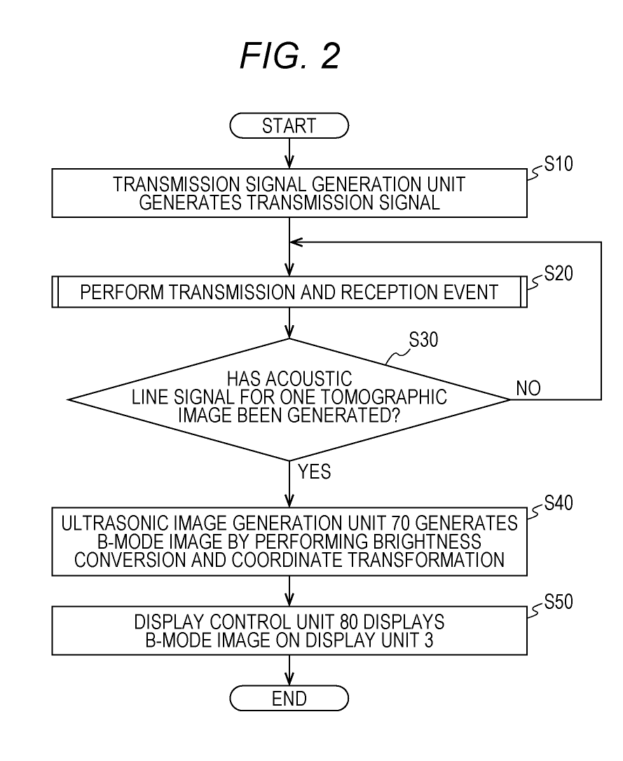

The operation of the ultrasonic diagnostic apparatus 1 according to the first embodiment will be described. FIG. 2 is a flowchart showing the operation of the ultrasonic diagnostic apparatus 1.

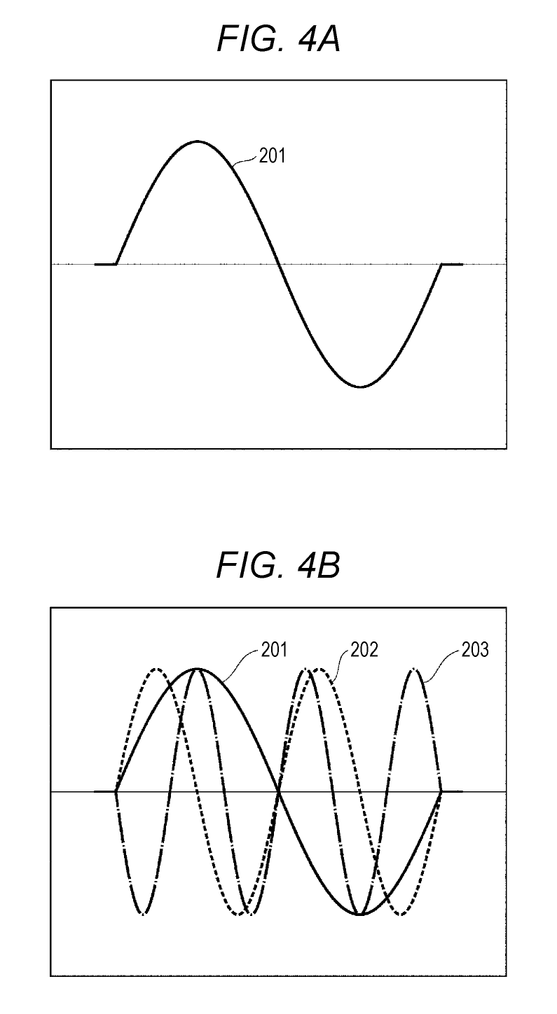

First, the transmission signal generation unit 10 generates a transmission signal (step S10). FIG. 4A shows an example of the waveform of a transmission pulse. A transmission pulse 201 shown in FIG. 4A is configured to include a fundamental wave component of one period. In addition, as shown in FIG. 4B, the transmission pulse may further include a pulse that has a frequency of integral multiples of the frequency of the fundamental wave component and that starts and ends simultaneously with the fundamental wave component. For example, the transmission pulse may further include a double pulse 202 and a triple pulse 203. In this case, it is preferable to generate a pulse having a frequency of an odd multiple of the frequency of the fundamental wave component so that the timing of the peak of the pulse matches that of the fundamental wave. In addition, the time length of the transmission pulse may not be one period of the fundamental wave component. For example, the time length of the transmission pulse may be other lengths, such as two periods of the fundamental wave component, and preferably one period or more of the fundamental wave component.

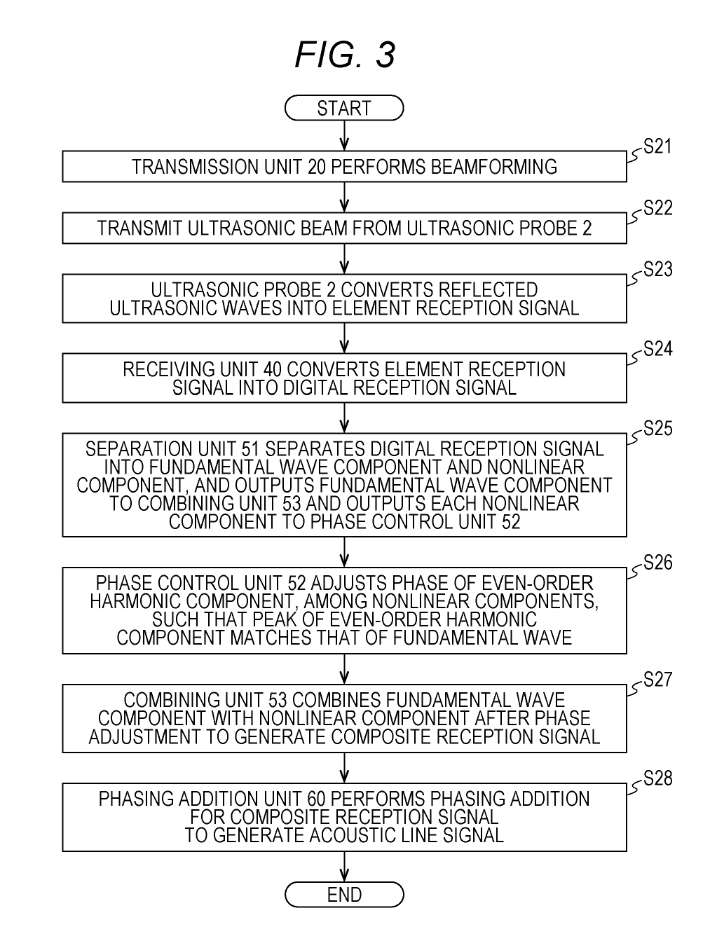

Then, a transmission and reception event is performed (step S20). Here, the transmission and reception event refers to a series of processing for transmitting ultrasonic waves to a subject based on the transmission signal and performing signal processing based on the reflected ultrasonic wave. FIG. 3 is a flowchart showing the details of the transmission and reception event. Hereinafter, the operation of the ultrasonic diagnostic apparatus 1 according to the transmission and reception event will be described with reference to FIG. 3.

First, the transmission unit 20 performs transmission beamforming (step S21). Specifically, as described above, an element driving signal is generated for each transducer by setting the delay time for each transducer for the transmission timing of the ultrasonic beam and delaying the transmission signal by the delay time. The transmission unit 20 transmits the generated element driving signal to each relevant transducer of the ultrasonic probe 2 through the switching unit 30.

Then, an ultrasonic beam is transmitted to the inside of the subject from the ultrasonic probe 2 (step S22). Specifically, as described above, since each transducer of the ultrasonic probe 2 converts the element driving signal corresponding to itself into an ultrasonic wave, an ultrasonic beam is transmitted to the inside of the subject so as to be in focus at the transmission focus point.

Then, the transmitted ultrasonic beam propagates through the subject. At this time, due to the nonlinearity of a body tissue, harmonic components of different orders are generated. In addition, in a case where pulses of the same frequency components as the harmonics are included in the ultrasonic beam, the pulses and the harmonic components strengthen each other. The ultrasonic beam and the harmonic components generated within the subject are reflected by the boundary of the acoustic impedance of the body tissue or the like to reach the ultrasonic probe 2 as reflected ultrasonic waves.

Then, the ultrasonic probe 2 converts the reflected ultrasonic waves obtained from the inside of the subject into an element reception signal (step S23). Specifically, as described above, each transducer of the ultrasonic probe 2 converts the reflected ultrasonic wave into an electrical signal, and transmits the electrical signal, as an element reception signal, to the receiving unit 40 through the switching unit 30.

Then, the receiving unit 40 converts the element reception signal into a digital reception signal (step S24). Specifically, the receiving unit 40 converts the element reception signal into a digital reception signal by performing amplification and A/D conversion of the element reception signal.

Then, the separation unit 51 separates the digital reception signal into a fundamental wave component and nonlinear components (step S25). Specifically, the digital reception signal is separated into a fundamental wave component, a second harmonic component, a third harmonic component, and the like using a band pass filter. The separation unit 51 outputs the fundamental wave component to the combining unit 53, and outputs each harmonic component forming the nonlinear component to the phase control unit 52.

Then, the phase control unit 52 performs phase control for the nonlinear component (step S26). The phase control unit 52 adjusts the phases of the second harmonic component, the third harmonic component, and the like so that the timing of the peak of each of the second harmonic component, the third harmonic component, and the like matches that of the fundamental wave component. Specifically, an odd harmonics group (the third harmonic component, the fifth harmonic component, and the like) is output as it is, and the phase of an even harmonics group (the second harmonic component, the fourth harmonic component, and the like) is delayed by π/2. In addition, here, as a method of adjusting the phase, the phase is delayed by a time corresponding to the phase to be delayed. For example, as a method of delaying the phase of the harmonic component of 8 MHz by π/2, the phase is delayed by {1/(8×10⁶)}×¼=31.25×10⁻⁹, that is, 31.25 ns.

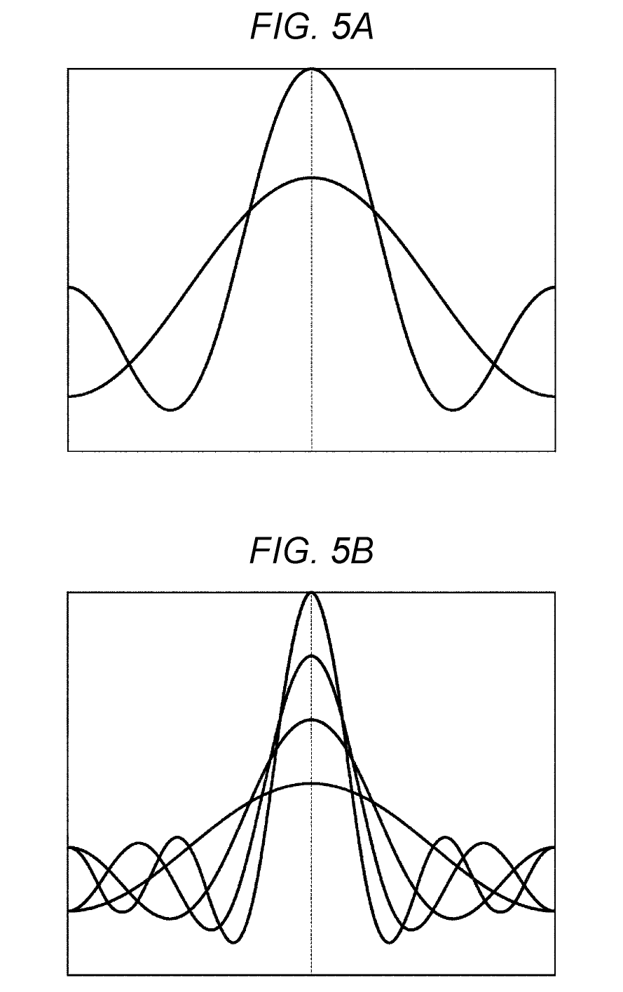

Then, the combining unit 53 combines the nonlinear component after the phase control with the fundamental wave to generate a composite reception signal (step S27). Specifically, the fundamental wave and the nonlinear component are combined at a predetermined combination ratio. Therefore, as shown in FIGS. 5A and 5B, since the fundamental wave and the nonlinear component timings of peaks of which match each other are combined, peaks become steep, and the substantial pulse width (for example, a full width at half maximum) is reduced. FIG. 5A shows a case of combining the fundamental wave and the second harmonic, and FIG. 5B shows a case of combining the fundamental wave and the second to fourth harmonics.

Then, the phasing addition unit 60 performs phasing addition for the composite reception signal to convert the composite reception signal into an acoustic line signal (step S28). The phasing addition unit 60 generates an acoustic line signal by performing delay processing on each composite reception signal so that the reception timing from the observation point is the same and adding the composite reception signals after the delay, for each observation point in a region for which an acoustic line signal is to be generated. Here, the observation point is a point, which is different from the transmission focus point and the transmission focus point only in depth, or the vicinity thereof.

As described above, one transmission and reception event is ended.

Referring back to FIG. 2, the explanation will be continued. Then, it is determined whether or not an acoustic line signal has been acquired for the entire region of interest for which a B-mode image is to be generated (step S30). In a case where there is a region for which an acoustic line signal has not been acquired, a position where the ultrasonic beam is transmitted is changed, and the transmission and reception event in step S20 is performed again to generate an acoustic line signal. On the other hand, in a case where an acoustic line signal has been generated for the entire region of interest for which a B-mode image is to be generated, the process proceeds to step S40.

Then, the ultrasonic image generation unit 70 generates a B-mode image by performing envelope detection, brightness conversion using logarithmic compression, and coordinate transformation to the orthogonal coordinate system for the acoustic line signal of the entire region of interest (step S40).

Finally, the display control unit 80 displays the B-mode image generated by the ultrasonic image generation unit 70 on the display unit 3 (step S50).

<Summary>

Through the configuration described above, since it is possible to make the peak of the composite reception signal steep without performing pulse compression using correlation processing, it is possible to substantially narrow the pulse width. Therefore, it is possible to improve the distance resolution of a B-mode image to be generated.

In addition, in a case where the nonlinear component is so small that it is not possible to make the peak of the composite reception signal steep, it is possible to generate a B-mode image using only the fundamental wave component even though it is not possible to take advantage of the THI, such as an improvement in S/N ratio. That is, a region that cannot be imaged by the THI can be imaged using the fundamental wave component. Accordingly, it is possible to obtain a so-called frequency compound effect of performing switching between improvements in the S/N ratio and resolution due to high-frequency ultrasonic waves and an improvement in penetration performance due to low-frequency ultrasonic waves appropriately according to the conditions, such as the depth.

### First Modification Example

In the first embodiment, the case of using a band pass filter when extracting nonlinear components has been described. In contrast, in this modification example, a case of extracting nonlinear components using a phase inversion method (hereinafter, also referred to as a “pulse inversion method”) will be described.

<Configuration>

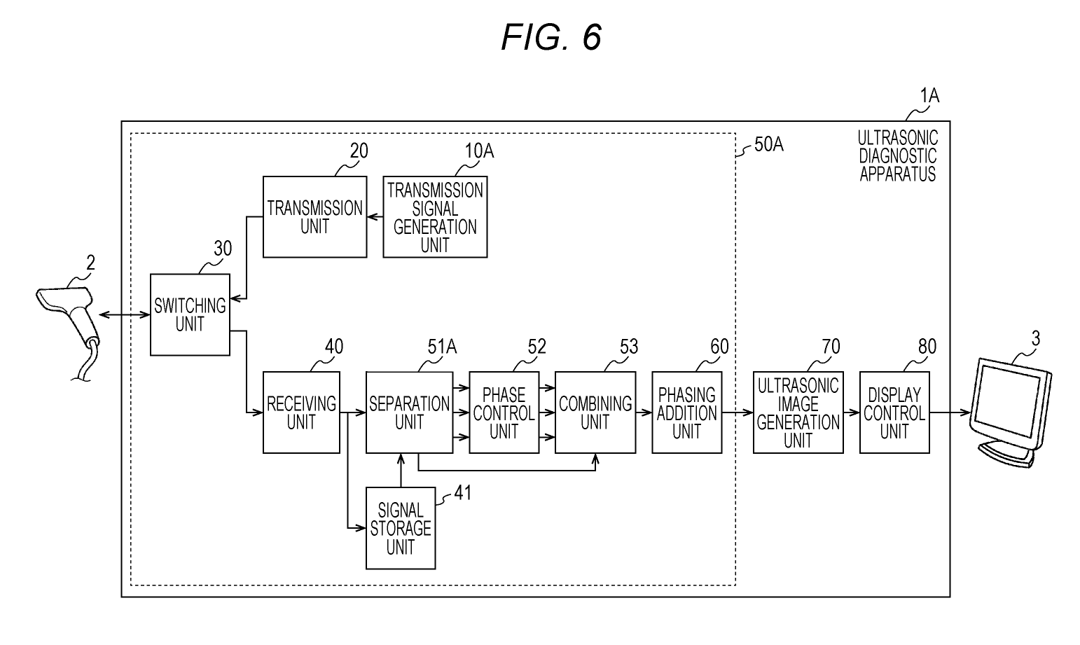

FIG. 6 shows a block diagram of an ultrasonic diagnostic apparatus 1A according to a first modification example. In addition, the same components as in FIG. 1 are denoted by the same reference numerals, and the explanation thereof will be omitted.

The ultrasonic diagnostic apparatus 1A is characterized in that a transmission signal generation unit 10A, a signal storage unit 41, and a separation unit 51A for extracting nonlinear components using the phase inversion method are provided, and other configurations are the same as those of the ultrasonic diagnostic apparatus 1. In addition, the transmission signal generation unit 10A, the transmission unit 20, the switching unit 30, the receiving unit 40, the signal storage unit 41, the separation unit 51A, the phase control unit 52, the combining unit 53, and the phasing addition unit 60 form an ultrasonic signal processing circuit 50A.

The signal storage unit 41 is a storage medium for storing a plurality of digital reception signals according to one transmission and reception event. Specifically, the signal storage unit 41 is realized by a memory or the like.

The separation unit 51A is a circuit for separating a digital reception signal into an even harmonics group, a fundamental wave component, and an odd harmonics group using the phase inversion method and then performing separation into respective components. The details thereof will be described later.

<Operation>

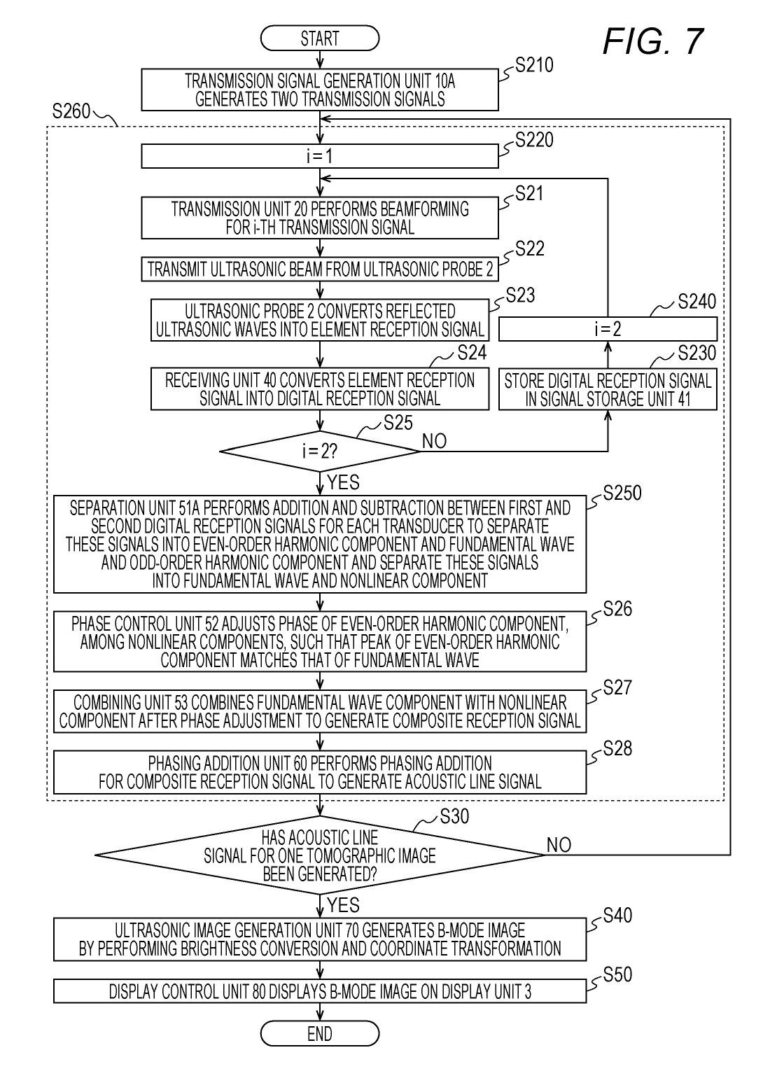

The operation of the ultrasonic diagnostic apparatus according to the first modification example will be described. FIG. 7 is a flowchart showing the operation of the ultrasonic diagnostic apparatus according to the first modification example. In addition, the same operations as in FIGS. 2 and 3 are denoted by the same step numbers, and the detailed explanation thereof will be omitted.

First, the transmission signal generation unit 10A generates transmission signals (step S210). Here, the transmission signal generation unit 10A generates two transmission signals. The first transmission signal is a transmission pulse shown in FIG. 4A or 4B that has been described in the first embodiment. On the other hand, the second transmission signal is a pulse obtained by inverting the phase of the fundamental wave and the phase of a pulse having a frequency of odd multiples of the frequency of the fundamental wave. That is, the first transmission pulse shown in FIG. 4A is a transmission pulse obtained by inverting the phase of the transmission pulse 201. In addition, the first transmission pulse shown in FIG. 4B is a transmission pulse obtained by inverting the phases of the transmission pulse 201 and the triple pulse 203. In this case, the phase of a pulse having a frequency of even multiples of the frequency of the fundamental wave, for example, the phase of the double pulse 202 is not inverted.

Then, a transmission and reception event (step S260) is performed.

First, ultrasonic waves are transmitted and received using the first transmission pulse (step S220). Then, the transmission unit 20 performs transmission beamforming (step S21). Then, an ultrasonic beam is transmitted to the inside of the subject from the ultrasonic probe 2 (step S22). Then, the reflected ultrasonic waves obtained from the inside of the subject by the ultrasonic probe 2 are converted into an element reception signal (step S23). Then, the receiving unit 40 converts each element reception signal into a digital reception signal (step S24). The generated digital reception signal is stored in the signal storage unit 41 (step S230).

Then, ultrasonic waves are transmitted and received using the second transmission pulse (step S240). First, the transmission unit 20 performs transmission beamforming (step S21). Then, an ultrasonic beam is transmitted to the inside of the subject from the ultrasonic probe 2 (step S22). Then, the reflected ultrasonic waves obtained from the inside of the subject by the ultrasonic probe 2 are converted into an element reception signal (step S23). Then, the receiving unit 40 converts each element reception signal into a digital reception signal (step S24). The generated digital reception signal is output to the separation unit 51A.

After finishing the transmission and reception of ultrasonic waves using both the first transmission pulse and the second transmission pulse (Yes in step S25), the separation unit 51A separates the digital reception signal into a fundamental wave component and a nonlinear component (step S250). First, the separation unit 51A reads the digital reception signal obtained by the first transmission pulse from the signal storage unit 41. Then, the separation unit 51A performs addition and subtraction between the digital reception signal obtained by the first transmission pulse and the digital reception signal obtained by the second transmission pulse, all of which have been obtained from the same transducer. In a case where the phase of the fundamental wave is inverted, the phases of the fundamental wave component and the odd harmonics group are inverted, but the phase of the even harmonics group is not inverted. Accordingly, when the digital reception signal obtained by the first transmission pulse and the digital reception signal obtained by the second transmission pulse are added up, the fundamental wave component and the odd harmonics group are canceled out since the phases are inverted with respect to each other. As a result, only the even harmonics group having the same phase is obtained. In addition, when the digital reception signal obtained by the second transmission pulse is subtracted from the digital reception signal obtained by the first transmission pulse, the even harmonics group is canceled out since the phases match each other. As a result, only the fundamental wave component and the odd harmonics group with phases inverted with respect to each other are obtained. By using a band pass filter for the even harmonics group, the fundamental wave component, and the odd harmonics group obtained as described above, the even harmonics group can be separated into the second harmonic component, the fourth harmonic component, and the like, and the fundamental wave component and the odd-order harmonic component can be separated into the fundamental wave component, the third harmonic component, the fifth harmonic component, and the like. The separation unit 51A outputs the fundamental wave component to the combining unit 53, and outputs each harmonic component to the phase control unit 52 as a nonlinear component.

Then, the phase control unit 52 performs phase control for the nonlinear component (step S26).

Then, the combining unit 53 combines the nonlinear component after the phase control with the fundamental wave to generate a composite reception signal (step S27).

Then, the phasing addition unit 60 performs phasing addition for the composite reception signal to convert the composite reception signal into an acoustic line signal (step S28).

Then, it is determined whether or not an acoustic line signal has been acquired for the entire region of interest for which a B-mode image is to be generated (step S30). In a case where there is a region for which an acoustic line signal has not been acquired, a position where the ultrasonic beam is transmitted is changed, and the transmission and reception event in step S260 is repeated to generate an acoustic line signal. On the other hand, in a case where an acoustic line signal has been generated for the entire region of interest for which a B-mode image is to be generated, the process proceeds to step S40.

Then, the ultrasonic image generation unit 70 generates a B-mode image by performing envelope detection, brightness conversion using logarithmic compression, and coordinate transformation to the orthogonal coordinate system for the acoustic line signal of the entire region of interest (step S40).

Finally, the display control unit 80 displays the B-mode image generated by the ultrasonic image generation unit 70 on the display unit 3 (step S50).

<Summary>

Through the configuration described above, even if there is an overlapping band between two components having frequencies adjacent to each other, for example, the fundamental wave component and the second harmonic component, if one of the two components is the fundamental wave component or belongs to the odd harmonics group and the other one belongs to the even harmonics group, one specific component can be separated without band loss and without other remaining components. Therefore, even in a state in which there is an overlapping band between a fundamental wave component and the second harmonic component and/or between the second harmonic component and the third harmonic component, it is possible to extract each component without band loss. As a result, it is possible to obtain a high-quality composite reception signal.

### Second Modification Example

In the first embodiment and the first modification example, the case where only one fundamental wave component is used has been described. In contrast, in this modification example, a case where a plurality of fundamental wave components are used will be described.

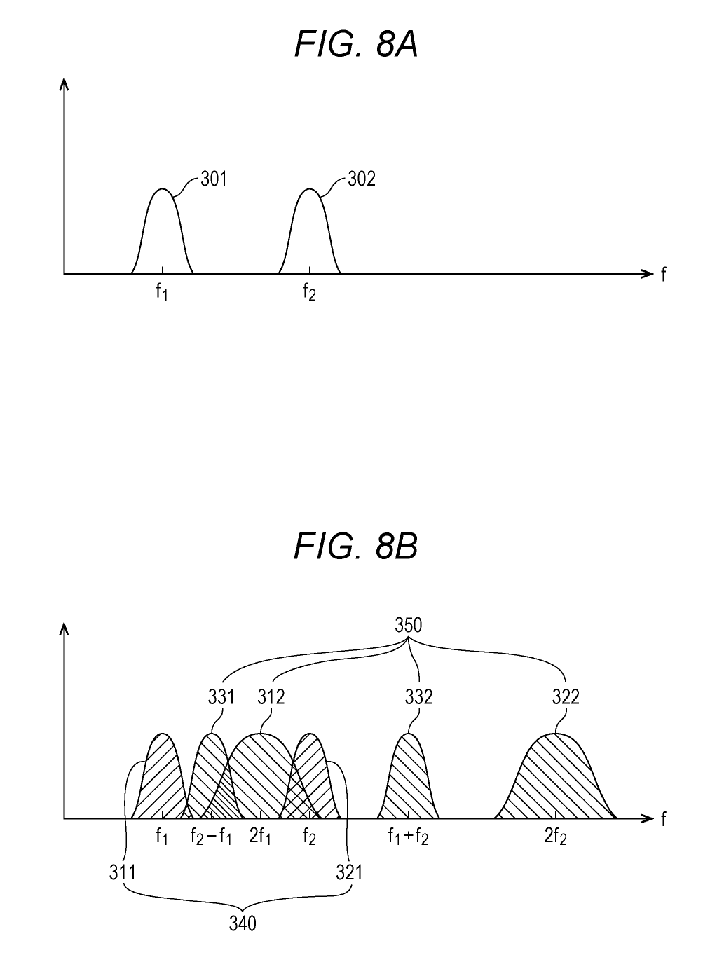

FIGS. 8A and 8B show the bands of a transmission ultrasonic pulse and a reception ultrasonic wave. As shown in FIG. 8A, the transmission ultrasonic pulse includes a fundamental wave 301 having a frequency f₁ and a fundamental wave 302 having a frequency f₂. In addition, it is preferable to transmit the transmission ultrasonic pulse so that the timings of the peaks of the fundamental wave 301 and the fundamental wave 302 match each other. On the other hand, as shown in FIG. 8B, the reception ultrasonic wave includes not only a fundamental wave component 311 having a frequency f₁ and a fundamental wave component 321 having a frequency f₂ but also a second harmonic component 312 having a frequency 2f₁, a second harmonic component 322 having a frequency 2f₂, a difference frequency component 331 having a frequency f₂−f₁, a sum frequency component 332 having a frequency f₁+f₂, and the like. In a case where the timings of the peaks of the fundamental wave 301 and the fundamental wave 302 match each other, the timings of the peaks of the fundamental wave component 311 and the fundamental wave component 321 belonging to a fundamental wave group 340 match each other. In addition, the timings of the peaks of the second harmonic component 312 and the second harmonic component 322 belonging to an even harmonics group 350 match each other. In addition, the timings of the peaks of the difference frequency component 331 and the sum frequency component 332 match the timings of the peaks of the second harmonic component 312 and the second harmonic component 322 belonging to the even harmonics group 350. That is, it can be regarded that the difference frequency component 331 and the sum frequency component 332 belong to the even harmonics group 350. Therefore, in a case where the timings of the peaks of the fundamental wave 301 and the fundamental wave 302 match each other, in the difference frequency component 331, the second harmonic component 322, and the fundamental wave component 321 frequency bands of which overlap each other, the difference frequency component 331 and the second harmonic component 322 strengthen each other. On the other hand, in the fundamental wave group 340 and the even harmonics group 350, the timings of the peaks do not match each other. Accordingly, since the phase of the fundamental wave component 321 does not match any of the phases of the difference frequency component 331 and the second harmonic component 322, the fundamental wave component 321 and the difference frequency component 331 and the second harmonic component 322 do not strengthen each other.

Therefore, using the same configuration as in the first embodiment or the first modification example, the second harmonic component 312, the second harmonic component 322, the difference frequency component 331, and the sum frequency component 332 that belong to the even harmonics group 350 are extracted by the separation unit, and the phases of these components are controlled by the phase control unit. Thus, since the timings of the peaks can be made to match each other in the fundamental wave group 340 and the even harmonics group 350, it is possible to make the peak steep.

<Summary>

Through the configuration described above, since it is possible to make a peak steep by making two arbitrary components having different frequencies strengthen each other, it is possible to achieve both an improvement in the use efficiency of ultrasonic waves and an improvement in signal quality.

### Third Modification Example

In the first embodiment and the first and second modification examples, the case has been described in which the separation unit outputs a fundamental wave component to the combining unit and outputs each component forming nonlinear components to the phase control unit and the phase control unit performs phase control only for even-order harmonic components among the nonlinear components. In contrast, in a third modification example, another embodiment regarding components to be subjected to phase control processing and its control method will be described.

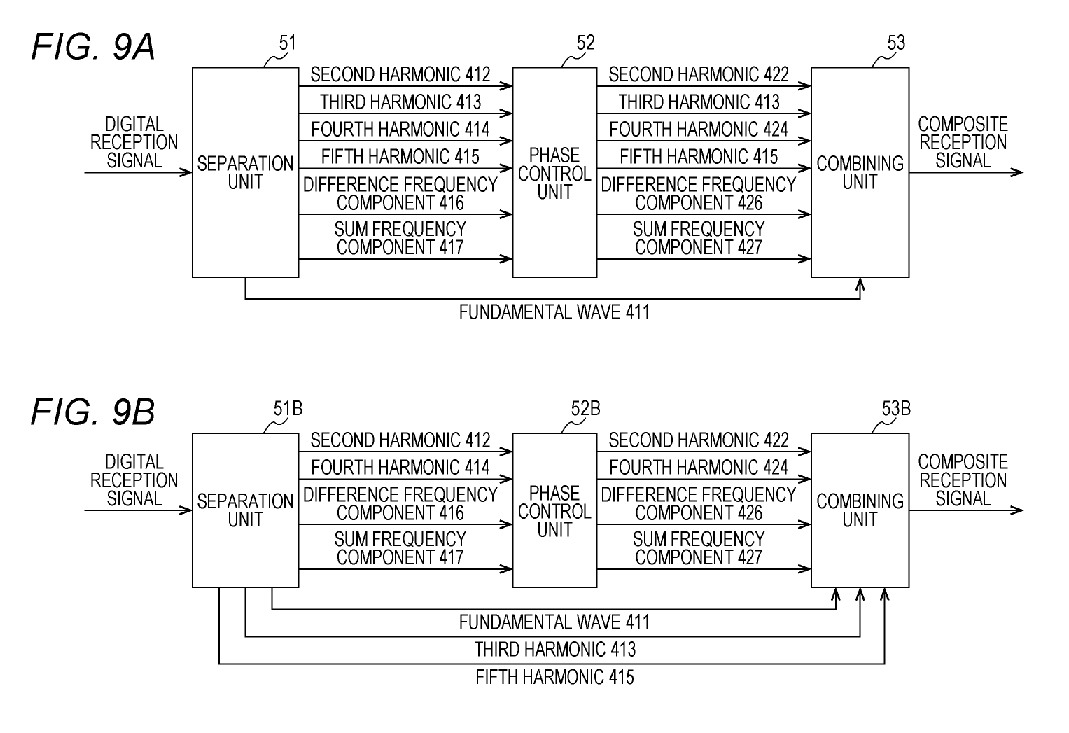

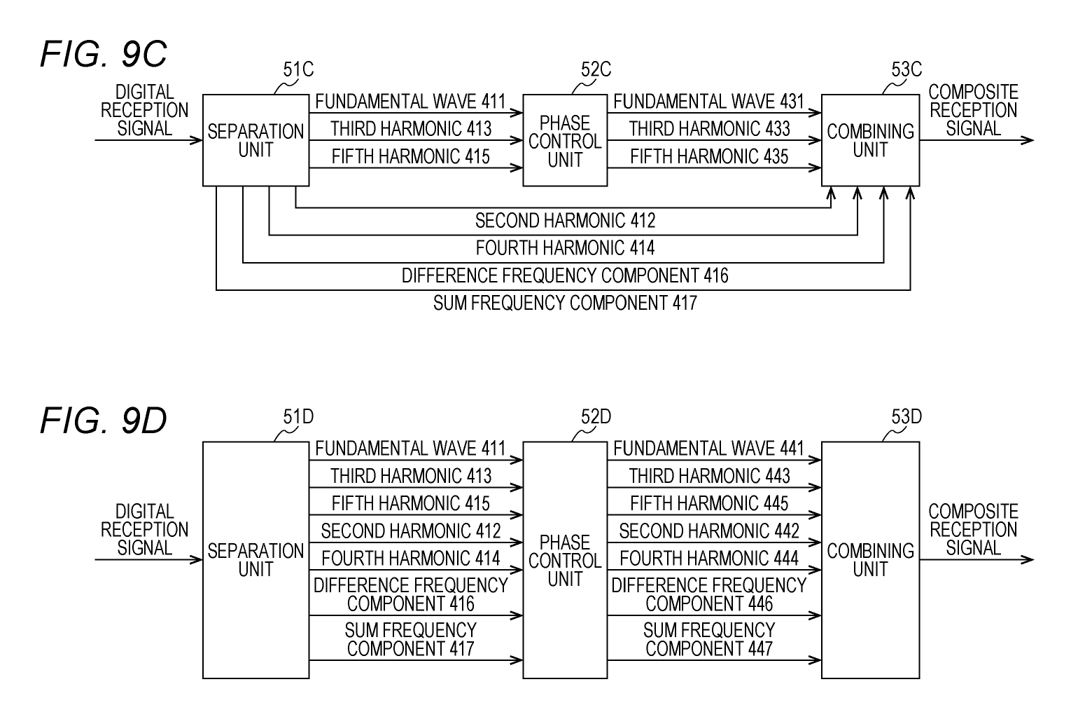

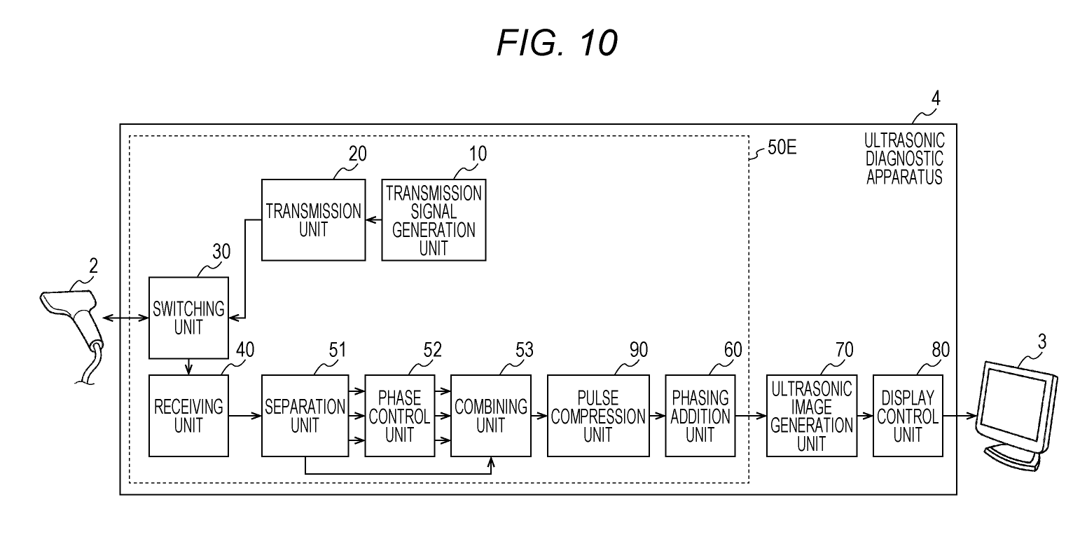

FIGS. 9A to 9D are schematic diagrams showing components to be subjected to frequency separation and phase control processing. In addition, each component shown in FIGS. 9A to 9D and the following explanation is just an example, and even-order harmonic components and odd-order harmonic components other than the described frequency components may be further used, or only some of the described even-order harmonic components and odd-order harmonic components may be used.

FIG. 9A shows a configuration for the separation and the phase control described in the first embodiment and the first and second modification examples. In this configuration, the separation unit 51 outputs a fundamental wave 411 to the combining unit 53 as it is, and outputs a second harmonic 412, a third harmonic 413, a fourth harmonic 414, a fifth harmonic 415, a difference frequency component 416, and a sum frequency component 417, which are nonlinear components, to the phase control unit 52. The phase control unit 52 allows the odd harmonics group to be transmitted as it is, and performs phase control for the even harmonics group. That is, the third harmonic 413 and the fifth harmonic 415 are transmitted through the phase control unit 52 as they are. On the other hand, the phase control unit 52 controls the phases of the second harmonic 412, the fourth harmonic 414, the difference frequency component 416, and the sum frequency component 417, and outputs a second harmonic 422, a fourth harmonic 424, a difference frequency component 426, and a sum frequency component 427 after the phase control to the combining unit 53. The combining unit 53 generates a composite reception signal by combining the respective components of the fundamental wave component, the odd harmonics group, and the even harmonics group after the phase control.

FIG. 9B shows a configuration for separation and phase control according to another embodiment. In this configuration, a separation unit 51B outputs the fundamental wave 411 and the third harmonic 413 and the fifth harmonic 415, which belong to the odd harmonics group, to a combining unit 53B as they are, and outputs the second harmonic 412 and the fourth harmonic 414 belonging to the even harmonics group, the difference frequency component 416, and the sum frequency component 417, among nonlinear components, to a phase control unit 52B. That is, since the timing of the peak of the fundamental wave component and the timing of the peak of each component of the odd harmonics group match each other, the fundamental wave component and each component of the odd harmonics group are directly output to the combining unit 53B. The phase control unit 52B controls the phases of the second harmonic 412 and the fourth harmonic 414 belonging to the even harmonics group, the difference frequency component 416, and the sum frequency component 417, which have been received, and outputs the second harmonic 422, the fourth harmonic 424, the difference frequency component 426, and the sum frequency component 427 after the phase control to the combining unit 53B. The combining unit 53B generates a composite reception signal by combining the respective components of the fundamental wave component, the odd harmonics group, and the even harmonics group after the phase control. In this manner, the odd harmonics group for which phase control is not required can be directly output from the separation unit 51B. In addition, the separation unit 51B may output the fundamental wave component and the odd harmonics group to the combining unit 53B in a state in which the fundamental wave component and the odd harmonics group are mixed, without separating the fundamental wave component and the odd harmonics group into respective components, such as the fundamental wave component, the third harmonic component, and the fifth harmonic component. Through the configuration, the separation unit 51B can output a signal, to which a filter that does not transmit only the even harmonics group has been applied, to the combining unit 53B as it is. In particular, in a case where the separation unit 51B uses the phase inversion method shown in the first modification example, the fundamental wave component and the odd harmonics group obtained by subtraction between the digital reception signal obtained by the first transmission pulse and the digital reception signal obtained by the second transmission pulse may be output to the combining unit 53B as they are. In this case, since it is not necessary to use a band pass filter by which a part of the band of the fundamental wave component and the odd harmonics group is lost, it is possible to eliminate the chance of band loss.

FIG. 9C shows a configuration for separation and phase control according to still another embodiment. In this configuration, a separation unit 51C outputs the second harmonic 412 and the fourth harmonic 414 belonging to the even harmonics group, the difference frequency component 416, and the sum frequency component 417 to a combining unit 53C as they are, and outputs the fundamental wave 411 and the third harmonic 413 and the fifth harmonic 415, which belong to the odd harmonics group, to a phase control unit 52C. That is, contrary to the configuration shown in FIG. 9B, the phase of the even harmonics group is not controlled, but the phase of each component belonging to the fundamental wave component and the odd harmonics group is controlled so that the timing of the peak matches that of each component of the even harmonics group. Specifically, for example, control to advance the phase of the fundamental wave component and the phase of each component belonging to the odd harmonics group by π/2 is performed. The phase control unit 52C controls the phases of the fundamental wave 411, the third harmonic 413, and the fifth harmonic 415, which have been received, and outputs a fundamental wave 431, a third harmonic 433, and a fifth harmonic 435 after the phase control to the combining unit 53C. The combining unit 53C generates a composite reception signal by combining the respective components of the even harmonics group and the fundamental wave component and the odd harmonics group after the phase control. In this manner, the even harmonics group for which phase control is not required can be directly output from the separation unit 51C. In this case, similar to the configuration shown in FIG. 9B, the separation unit 51C may output the even harmonics group to the combining unit 53C in a mixed state without separating the even harmonics group into respective components. Through the configuration, the separation unit 51C can output a signal, to which a filter that does not transmit the fundamental wave component and the odd harmonics group has been applied, to the combining unit 53C as it is. In particular, in a case where the separation unit 51C uses the phase inversion method shown in the first modification example, the even harmonics group obtained by addition between the digital reception signal obtained by the first transmission pulse and the digital reception signal obtained by the second transmission pulse may be output to the combining unit 53C as it is. In this case, since it is not necessary to use a band pass filter by which a part of the band of the even harmonics group is lost, it is possible to eliminate the chance of band loss.

FIG. 9D shows a configuration for separation and phase control according to still another embodiment. In this configuration, a separation unit 51D outputs the fundamental wave 411 and the third harmonic 413 and the fifth harmonic 415, which are odd-order harmonic components among the nonlinear components, to a phase control unit 52D, and outputs the second harmonic 412 and the fourth harmonic 414 that are even-order harmonic components among the nonlinear components, the difference frequency component 416, and the sum frequency component 417 to the phase control unit 52D. That is, all of the components are output to the phase control unit. The phase control unit 52D performs phase control for all of the received components. Here, by adjusting the amount of control of the phases of the fundamental wave component and the odd-order harmonic component and the amount of control of the phase of the even-order harmonic component, the timings of the peaks of the fundamental wave component and the odd-order harmonic component are made to match the timing of the peak of the even-order harmonic component. For example, by advancing the phases of the fundamental wave component and the odd-order harmonic component by π/4 and delaying the phase of the even-order harmonic component by π/4, it is possible to match the timings of the peaks of the fundamental wave component and the odd-order harmonic component with the timing of the peak of the even-order harmonic component. In addition, the amount of control of the phase is not limited to the example described above. For example, the phases of the fundamental wave component and the odd-order harmonic component may be advanced by π/3 and the phase of the even-order harmonic component may be delayed by 2π/3, or the phases of the fundamental wave component and the odd-order harmonic component may be advanced by π/2 and the amount of control of the phase of the even-order harmonic component may be set to 0 (that is, the even-order harmonic component is output as it is without phase control). That is, the amount of control of the phase may be arbitrarily selected as long as the timings of the peaks of the fundamental wave component and the odd-order harmonic component match the timing of the peak of the even-order harmonic component. The phase control unit 52D outputs the fundamental wave 411 that is a fundamental wave component after the phase control and the odd-order harmonic component after the phase control, that is, a fundamental wave 441, a third harmonic 443, a fifth harmonic 445, even-order harmonic components after phase control (that is, a second harmonic 442 and a fourth harmonic 444), a difference frequency component 446, and a sum frequency component 447 to a combining unit 53D. The combining unit 53D generates a composite reception signal by combining all of the components after the phase control.

### Second Embodiment

In the first embodiment, the configuration of improving the distance resolution by narrowing the pulse has been described. In contrast, a second embodiment is characterized in that the effect of improving the distance resolution is enhanced by further performing pulse compression.

<Configuration>

FIG. 10 shows a block diagram of an ultrasonic diagnostic apparatus 4 according to the second embodiment. In addition, the same components as in FIG. 1 are denoted by the same reference numerals, and the explanation thereof will be omitted.

The ultrasonic diagnostic apparatus 4 includes a pulse compression unit 90 that performs pulse compression for a composite reception signal. The ultrasonic diagnostic apparatus 4 is characterized in that pulse compression is further performed for the composite reception signal, and other configurations are the same as those of the ultrasonic diagnostic apparatus 1. In addition, the transmission signal generation unit 10, the transmission unit 20, the switching unit 30, the receiving unit 40, the separation unit 51, the phase control unit 52, the combining unit 53, the pulse compression unit 90, and the phasing addition unit 60 form an ultrasonic signal processing circuit 50E.

The pulse compression unit 90 is a circuit for receiving a composite reception signal from the combining unit, generating a time-series signal by performing correlation processing between the composite reception signal and the reference signal, and outputting the time-series signal to the phasing addition unit 60. Here, the reference signal is obtained by adding a component, which has a frequency of an integral multiple of the frequency of a fundamental wave component of the transmission pulse generated by the transmission signal generation unit 10, to the fundamental wave component so that the timings of the peaks match each other. The pulse compression unit associates a time difference between the composite reception signal and the reference signal with a cross-correlation value between the composite reception signal and the reference signal, and outputs the result as a time-series signal.

<Operation>

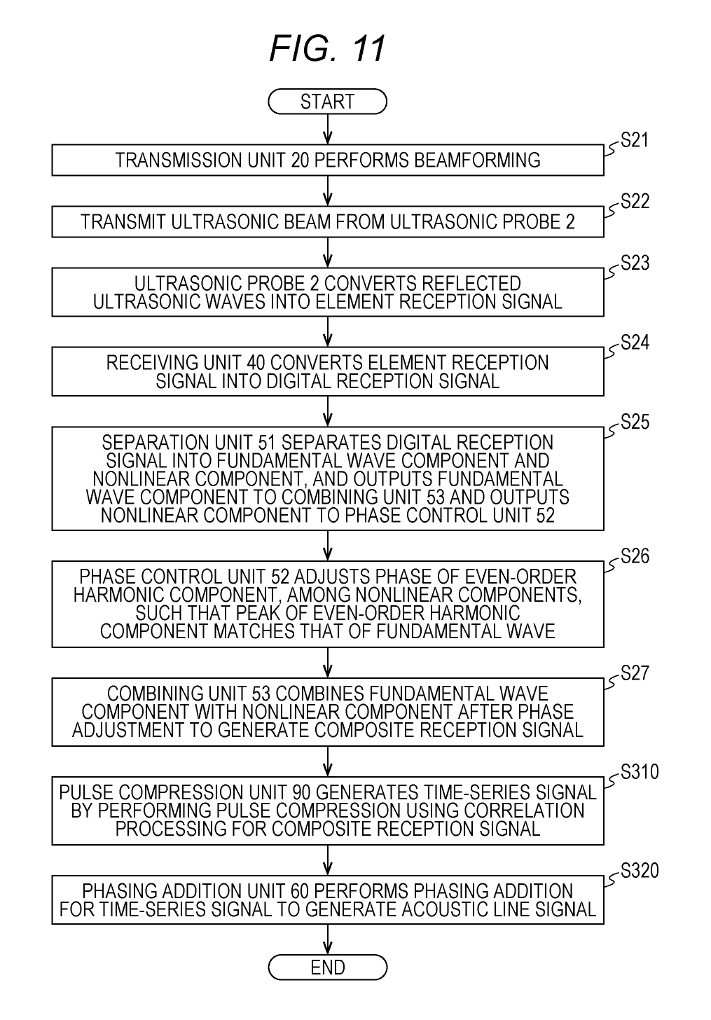

The operation of the ultrasonic diagnostic apparatus 4 according to the second embodiment will be described. The operation of ultrasonic diagnostic apparatus 4 is characterized in that the contents of the transmission and reception event are different, and operations other than the transmission and reception event are the same as those of the ultrasonic diagnostic apparatus 1. Hereinafter, the transmission and reception event will be described. FIG. 11 is a flowchart showing the operation of the transmission and reception event in the ultrasonic diagnostic apparatus 4. In addition, the same operations as in FIG. 3 are denoted by the same step numbers, and the detailed explanation thereof will be omitted.

First, the transmission unit 20 performs transmission beamforming (step S21).

Then, an ultrasonic beam is transmitted to the inside of the subject from the ultrasonic probe 2 (step S22).

Then, the reflected ultrasonic waves obtained from the inside of the subject by the ultrasonic probe 2 are converted into an element reception signal (step S23).

Then, the receiving unit 40 converts each element reception signal into a digital reception signal (step S24).

Then, the separation unit 51 separates the digital reception signal into a fundamental wave component and nonlinear components (step S25).

Then, the phase control unit 52 performs phase control for the nonlinear component (step S26).

Then, the combining unit 53 combines the nonlinear component after the phase control with the fundamental wave to generate a composite reception signal (step S27).

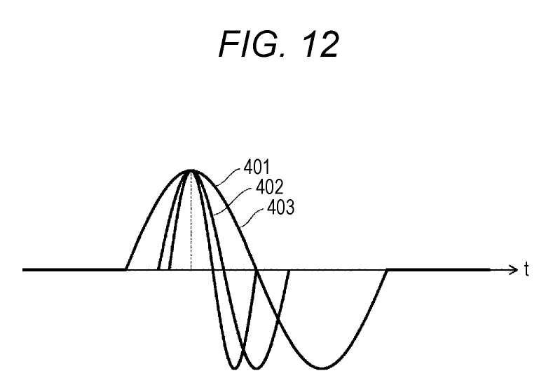

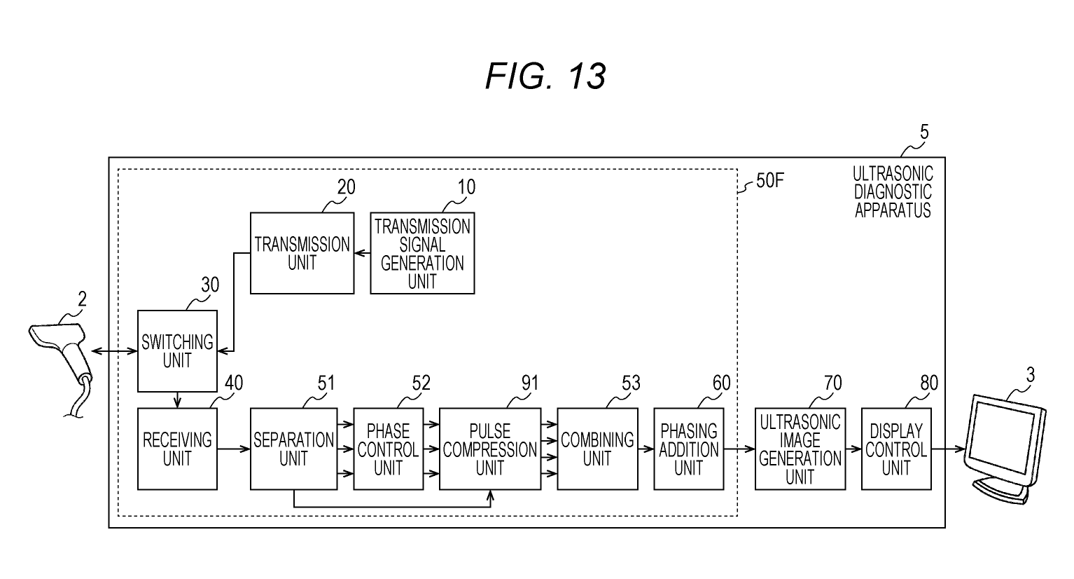

Then, the pulse compression unit 90 generates a time-series signal by performing correlation processing between the composite reception signal and the reference signal, and outputs the time-series signal to the phasing addition unit 60 (step S310). As described above, the reference signal used in the correlation processing is obtained by adding a component, which has a frequency of an integral multiple of the frequency of a fundamental wave component of the transmission pulse generated by the transmission signal generation unit 10, to the fundamental wave component so that the timings of the peaks match each other. Specifically, as shown in FIG. 12, the reference signal used in the correlation processing is obtained by adding a double pulse 402 and a triple pulse 403, each of which has a frequency of an integral multiple of the frequency of the same fundamental wave pulse 401 as a transmission signal, to the fundamental wave pulse 401. In addition, the timings of the peaks of the double pulse 402 and the triple pulse 403 are the same as the timing of the peak of the fundamental wave pulse 401. In addition, the reference signal may further include a quadruple pulse, a quintuple pulse, and the like. The pulse compression unit 90 calculates a cross-correlation value between the composite reception signal and the reference signal while changing the time difference between the composite reception signal and the reference signal. Finally, the pulse compression unit 90 generates a time-series signal by associating the cross-correlation value with the time difference between the composite reception signal and the reference signal, and outputs the time-series signal to the phasing addition unit 60.

Finally, the phasing addition unit 60 performs phasing addition for the time-series signal to generate an acoustic line signal (step S320).

In addition, although separation into respective components and phase control are the same as those in the first embodiment herein, the configurations of the first to third modification examples may be applied. For example, separation into respective components may be performed using the phase inversion method, and odd-order harmonic components may be directly output to the combining unit 53. In addition, only the fundamental wave component and the odd harmonics group or both of the even harmonics group and the fundamental wave component and the odd harmonics group may be subjected to phase control processing.

<Summary>

Through the configuration described above, since the pulse compression using correlation processing can be further performed for the composite reception signal whose substantial pulse length has been reduced by making the pulse steep, it is possible to further enhance the pulse compression effect. Therefore, it is possible to improve the distance resolution more reliably.

### Third Embodiment

In the second embodiment, a configuration has been described in which the effect of improving the distance resolution is further enhanced by narrowing the pulse by combining a plurality of frequency components and then performing pulse compression using correlation processing. In contrast, in the third embodiment, a case of performing pulse compression using correlation processing after phase control and then performing combination will be described.

<Configuration>

FIG. 13 shows a block diagram of an ultrasonic diagnostic apparatus 5 according to the third embodiment. In addition, the same components as in FIG. 1 are denoted by the same reference numerals, and the explanation thereof will be omitted.

The ultrasonic diagnostic apparatus 5 is characterized in that a pulse compression unit 91, which performs pulse compression for a fundamental wave component output from the separation unit 51 and nonlinear components after phase control output from the phase control unit 52, is provided and respective components after compression are combined, and other configurations are the same as those of the ultrasonic diagnostic apparatus 1. In addition, the transmission signal generation unit 10, the transmission unit 20, the switching unit 30, the receiving unit 40, the separation unit 51, the phase control unit 52, the pulse compression unit 91, the combining unit 53, and the phasing addition unit 60 form an ultrasonic signal processing circuit 50F.

The pulse compression unit 91 is a circuit for receiving a fundamental wave component from the separation unit 51 and receiving nonlinear components after phase control from the phase control unit 52, generating a time-series signal by performing correlation processing between each of the fundamental wave component and the nonlinear components and the reference signal, and outputting the time-series signal to the combining unit 53. Here, the reference signal is a fundamental wave component of the transmission pulse generated by the transmission signal generation unit 10 or a signal having the same frequency as a component to be subjected to correlation processing among components the timings of the peaks of which match that of the fundamental wave component and each of which has a frequency of an integral multiple of the frequency of the fundamental wave component. That is, for the fundamental wave component, the fundamental wave component of the transmission pulse generated by the transmission signal generation unit 10 is used as a reference signal. For the second harmonic component, a component, the timing of the peak of which matches that of the transmission pulse generated by the transmission signal generation unit 10 and which has a frequency that is twice the frequency of the fundamental wave component, is used as a reference signal. The pulse compression unit outputs a time difference between each component and the reference signal and a cross-correlation value between the composite component and the reference signal as a time-series component signal.

<Operation>

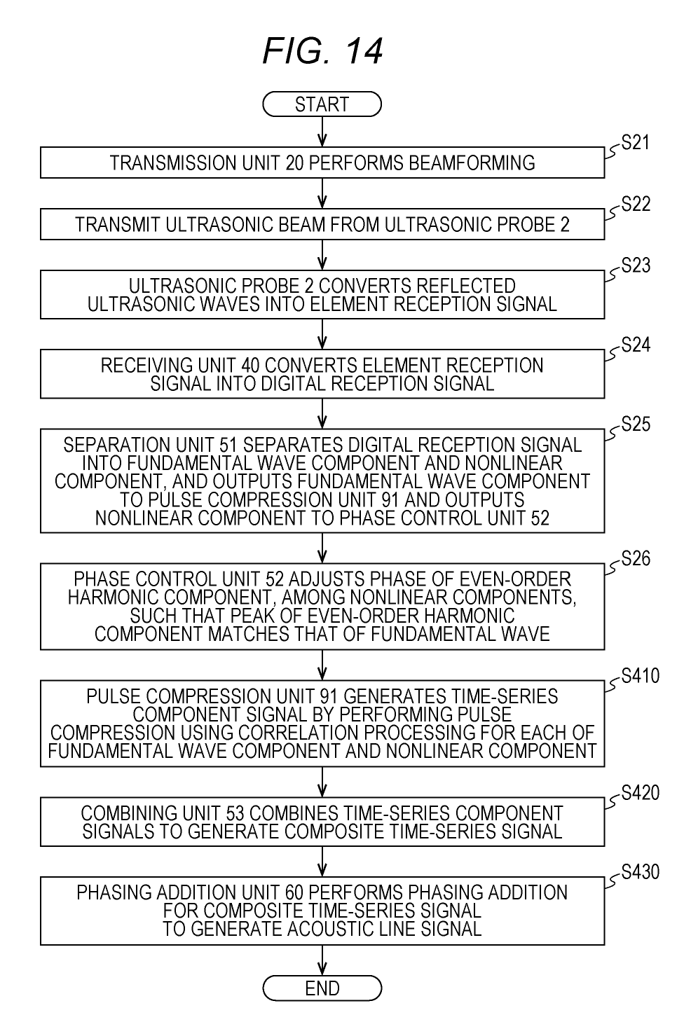

The operation of the ultrasonic diagnostic apparatus 5 according to the third embodiment will be described. The operation of ultrasonic diagnostic apparatus 5 is characterized in that the contents of the transmission and reception event are different, and operations other than the transmission and reception event are the same as those of the ultrasonic diagnostic apparatus 1. Hereinafter, the transmission and reception event will be described. FIG. 14 is a flowchart showing the operation of the transmission and reception event in the ultrasonic diagnostic apparatus 5. In addition, the same operations as in FIG. 3 are denoted by the same step numbers, and the detailed explanation thereof will be omitted.

First, the transmission unit 20 performs transmission beamforming (step S21).

Then, an ultrasonic beam is transmitted to the inside of the subject from the ultrasonic probe 2 (step S22).

Then, the reflected ultrasonic waves obtained from the inside of the subject by the ultrasonic probe 2 are converted into an element reception signal (step S23).

Then, the receiving unit 40 converts each element reception signal into a digital reception signal (step S24).

Then, the separation unit 51 separates the digital reception signal into a fundamental wave component and nonlinear components (step S25).

Then, the phase control unit 52 performs phase control for the nonlinear component (step S26).

Then, the pulse compression unit 91 generates a time-series component signal by performing correlation processing between the fundamental wave component and the nonlinear component after the phase control, and outputs the time-series component signal to the combining unit 53 (step S410). Here, as the reference signal, a fundamental wave component of the transmission pulse generated by the transmission signal generation unit 10 or a signal having the same frequency as a component to be subjected to correlation processing, among components the timings of the peaks of which match that of the fundamental wave component and each of which has a frequency of an integral multiple of the frequency of the fundamental wave component, is used. Specifically, for the fundamental wave component, as shown in FIG. 12, the same fundamental wave pulse 401 as a transmission signal is used. In addition, for the second harmonic component, the double pulse 402 is used. Similarly, for the third harmonic component, the triple pulse 403 is used. The pulse compression unit 91 calculates a cross-correlation value while changing the time difference between each of the fundamental wave component and the nonlinear component after phase control and the corresponding reference signal. Finally, the pulse compression unit 91 generates a time-series component signal by associating the cross-correlation value with the time difference between the composite reception signal and the reference signal for each component, and outputs the time-series component signal to the combining unit 53.

The combining unit 53 combines the time-series component signals to generate a composite time-series signal (step S420).

Finally, the phasing addition unit 60 performs phasing addition for the composite time-series signal to generate an acoustic line signal (step S430).

In addition, although separation into respective components and phase control are the same as those in the first embodiment herein, the configurations of the first to third modification examples may be applied. For example, separation into respective components may be performed using the phase inversion method, and odd-order harmonic components may be directly output to the combining unit 53. In addition, only the fundamental wave component and the odd harmonics group or both of the even harmonics group and the fundamental wave component and the odd harmonics group may be subjected to phase control processing.

<Summary>

Through the configuration described above, since the pulse compression of the fundamental wave component and each harmonic component, the phases of which have been controlled so that the timings of the peaks match each other, is performed by correlation processing, the timings of the peaks of time-series component signals generated from the fundamental wave component and the respective harmonic component signals match each other. For this reason, the peak of the composite time-series signal becomes steep. Therefore, since it is possible to greatly enhance the pulse compression effect, it is possible to improve the distance resolution more reliably.

### Fourth Embodiment

The configuration of performing only phase control for the nonlinear component has been described in the first embodiment, while the configuration of performing pulse compression by performing correlation processing after phase control has been described in the second and third embodiments. In contrast, in a fourth embodiment, a case of performing phase control after estimating and correcting the waveform of the nonlinear component will be described.

<Configuration>

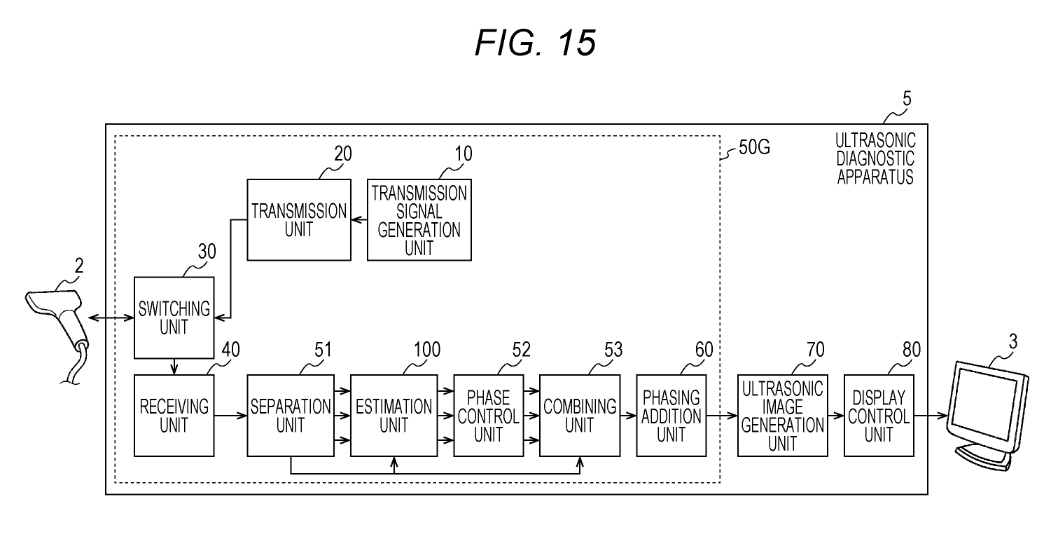

FIG. 15 shows a block diagram of an ultrasonic diagnostic apparatus 6 according to the fourth embodiment. In addition, the same components as in FIG. 1 are denoted by the same reference numerals, and the explanation thereof will be omitted.

The ultrasonic diagnostic apparatus 6 is characterized in that an estimation unit 100, which estimates and corrects the waveform of the nonlinear component using a fundamental wave component, is provided, and other configurations are the same as those of the ultrasonic diagnostic apparatus 1. In addition, the transmission signal generation unit 10, the transmission unit 20, the switching unit 30, the receiving unit 40, the separation unit 51, the estimation unit 100, the phase control unit 52, the combining unit 53, and the phasing addition unit 60 form an ultrasonic signal processing circuit 50G.

The estimation unit 100 is a circuit for receiving a fundamental wave component and a nonlinear component from the separation unit 51, estimating and correcting the waveform of the nonlinear component using the fundamental wave component, and outputting the nonlinear component after the correction to the phase control unit 52. The estimation unit 100 performs, for example, estimation processing using Bayesian statistics for each nonlinear component. More specifically, a nonlinear component is estimated and corrected based on the fundamental wave component using an inverse filter of noise, such as a Wiener filter.

<Operation>

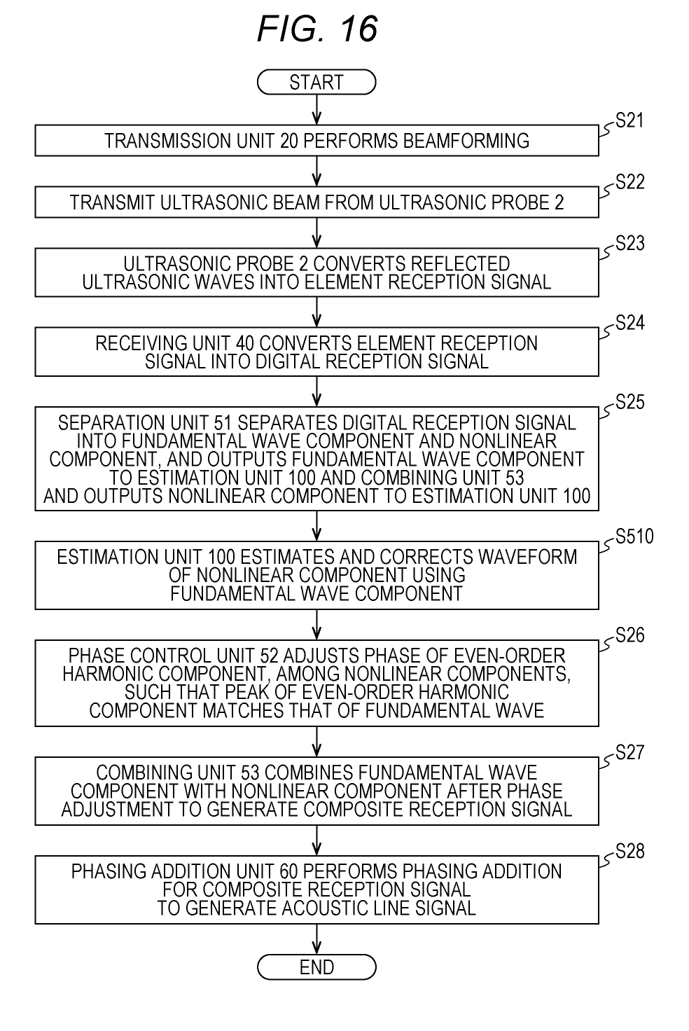

The operation of the ultrasonic diagnostic apparatus 6 according to the fourth embodiment will be described. The operation of ultrasonic diagnostic apparatus 6 is characterized in that the contents of the transmission and reception event are different, and operations other than the transmission and reception event are the same as those of the ultrasonic diagnostic apparatus 1. Hereinafter, the transmission and reception event will be described. FIG. 16 is a flowchart showing the operation of the transmission and reception event in the ultrasonic diagnostic apparatus 6. In addition, the same operations as in FIG. 3 are denoted by the same step numbers, and the detailed explanation thereof will be omitted.

First, the transmission unit 20 performs transmission beamforming (step S21).

Then, an ultrasonic beam is transmitted to the inside of the subject from the ultrasonic probe 2 (step S22).

Then, the reflected ultrasonic waves obtained from the inside of the subject by the ultrasonic probe 2 are converted into an element reception signal (step S23).

Then, the receiving unit 40 converts each element reception signal into a digital reception signal (step S24).

Then, the separation unit 51 separates the digital reception signal into a fundamental wave component and nonlinear components (step S25).

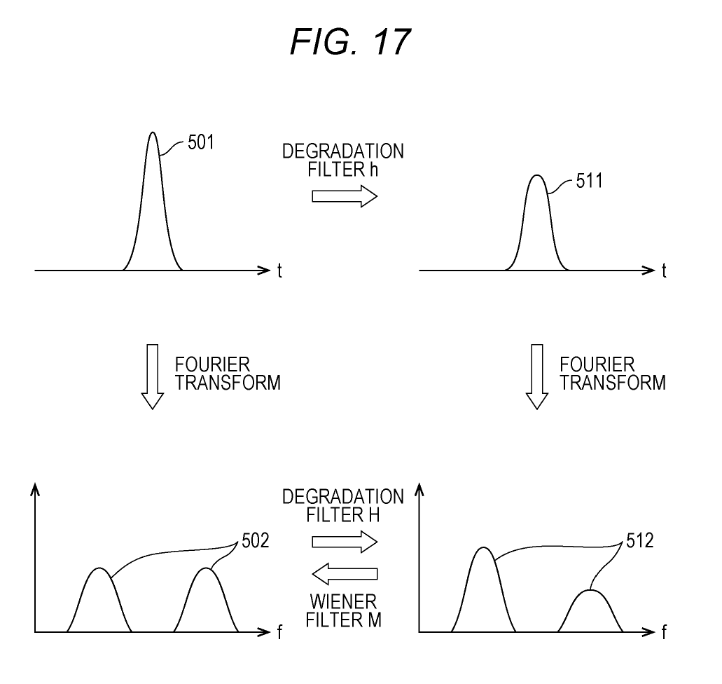

Then, the estimation unit 100 receives the fundamental wave component and the nonlinear component from the separation unit 51, estimates and corrects the waveform of the nonlinear component using the fundamental wave component, and outputs the nonlinear component after the correction to the phase control unit 52 (step S510). Estimation correction is performed by applying an inverse filter to the nonlinear component of the digital reception signal by regarding degradation until the nonlinear component reflected from the inside of the subject becomes a digital reception signal as the application of a degradation filter. FIG. 17 shows the schematic diagram. For example, it is assumed that a virtual digital reception signal 501 corresponding to the ultrasonic wave before degradation has become a digital reception signal 511 due to a degradation filter h. In this case, when a virtual frequency axis signal 502 and a frequency axis signal 512 obtained by performing a Fourier transform of the virtual digital reception signal 501 and the digital reception signal 511 are assumed, it can be assumed that the virtual frequency axis signal 502 has become the frequency axis signal 512 due to the degradation filter H. Therefore, if a Wiener filter M that is an inverse filter of the degradation filter H is applied to the frequency axis signal 512, the virtual frequency axis signal 502 is obtained. Specifically, the estimation unit 100 performs a Fourier transform of a composite signal of the fundamental wave component and the nonlinear component, calculates the Wiener filter M, which is an inverse filter of the degradation filter H, from the degradation model of the nonlinear component and applies the Wiener filter M, and extracts only the band of the nonlinear component by performing an inverse Fourier transform, thereby performing estimation reproduction.

Then, the phase control unit 52 performs phase control for the nonlinear component after the estimation correction (step S26).

Then, the combining unit 53 combines the nonlinear component after the phase control with the fundamental wave to generate a composite reception signal (step S27).

Finally, the phasing addition unit 60 performs phasing addition for the composite reception signal to convert the composite reception signal into an acoustic line signal (step S28).

In addition, although separation into respective components and phase control are the same as those in the first embodiment herein, the configurations of the first to third modification examples may be applied. For example, separation into respective components may be performed using the phase inversion method. In addition, odd-order harmonic components, among the nonlinear components estimated and corrected by the estimation unit 100, may be directly output to the combining unit 53. In addition, among the nonlinear components estimated and corrected by the estimation unit 100, only the fundamental wave component and the odd harmonics group or both of the even harmonics group and the fundamental wave component and the odd harmonics group may be subjected to phase control processing. In a case where the fundamental wave component and the odd harmonics group are phase control targets, the separation unit 51 may output the fundamental wave component to the estimation unit 100 and the phase control unit 52. Alternatively, the separation unit 51 may output the fundamental wave component only to the estimation unit 100, and the estimation unit 100 may allow the transmission of the fundamental wave component or may also perform an inverse Fourier transform for the band of the fundamental wave component at the time of estimation reproduction. In addition, for the odd harmonics group or the even harmonics group estimated and corrected by the estimation unit 100 that is not a phase control target, the odd harmonics group or the even harmonics group may be output to the combining unit 53 in a state in which all components of the odd harmonics group or all components of the even harmonics group are combined.

In addition, pulse compression using correlation processing may be performed for the composite reception signal or each component after phase control (after estimation reproduction for a component that is not a phase control target) by applying the second or third embodiment.

<Summary>

Through the configuration described above, since the nonlinear component is restored to the extent that the signal quality is not degraded by the estimation reproduction, it is possible to amplify the nonlinear component while maintaining the signal quality. Therefore, since it is possible to greatly enhance the pulse compression effect without amplifying noise, it is possible to greatly improve the distance resolution without quality degradation.

### Fifth Embodiment

In the first to fourth embodiments and each modification example, the case has been described in which the composite reception signal or the composite time-series signal is generated by performing component separation and phase control for the digital reception signal and the acoustic line signal is generated by performing phasing addition for the composite reception signal or the composite time-series signal. In contrast, in a fifth embodiment, a case will be described in which an acoustic line signal is generated by performing phasing addition for a digital reception signal and then a composite reception signal is generated by performing component separation and phase control for the acoustic line signal.

<Configuration>

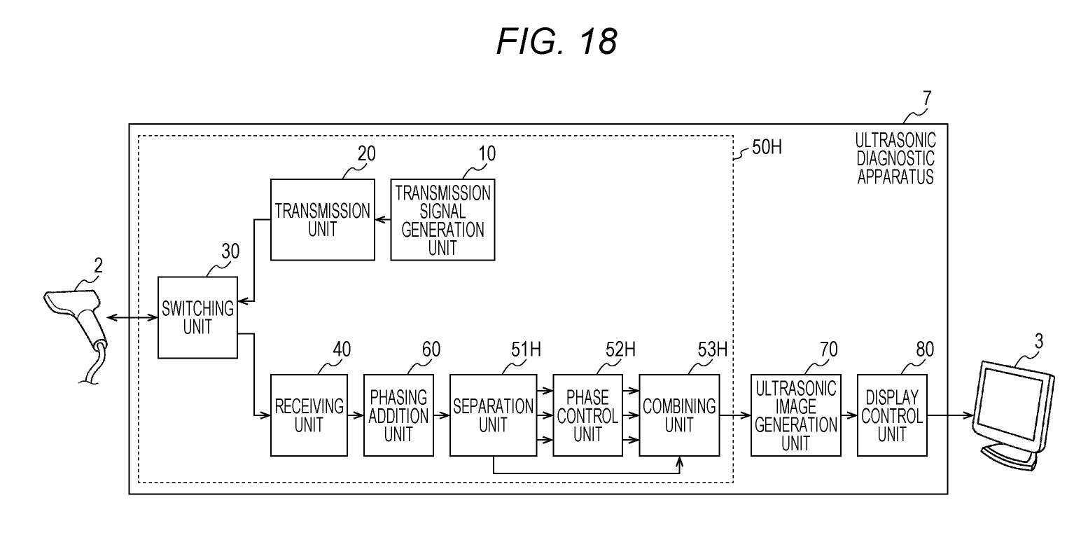

FIG. 18 shows a block diagram of an ultrasonic diagnostic apparatus 7 according to the fifth embodiment. In addition, the same components as in FIG. 1 are denoted by the same reference numerals, and the explanation thereof will be omitted.

The ultrasonic diagnostic apparatus 7 is characterized in that the phasing addition unit 60 is provided after the receiving unit 40 and before a separation unit 51H, and other configurations are the same as those of the ultrasonic diagnostic apparatus 1. In addition, the transmission signal generation unit 10, the transmission unit 20, the switching unit 30, the receiving unit 40, the phasing addition unit 60, the separation unit 51H, a phase control unit 52H, and a combining unit 53H form an ultrasonic signal processing circuit 50H.

The separation unit 51H, the phase control unit 52H, and the combining unit 53H are characterized in that the separation, phase control, and combination of the respective components of the acoustic line signal instead of the respective components of the digital reception signal are performed, and have the same configurations as the separation unit 51, the phase control unit 52, and the combining unit 53 except for that described above.

<Operation>

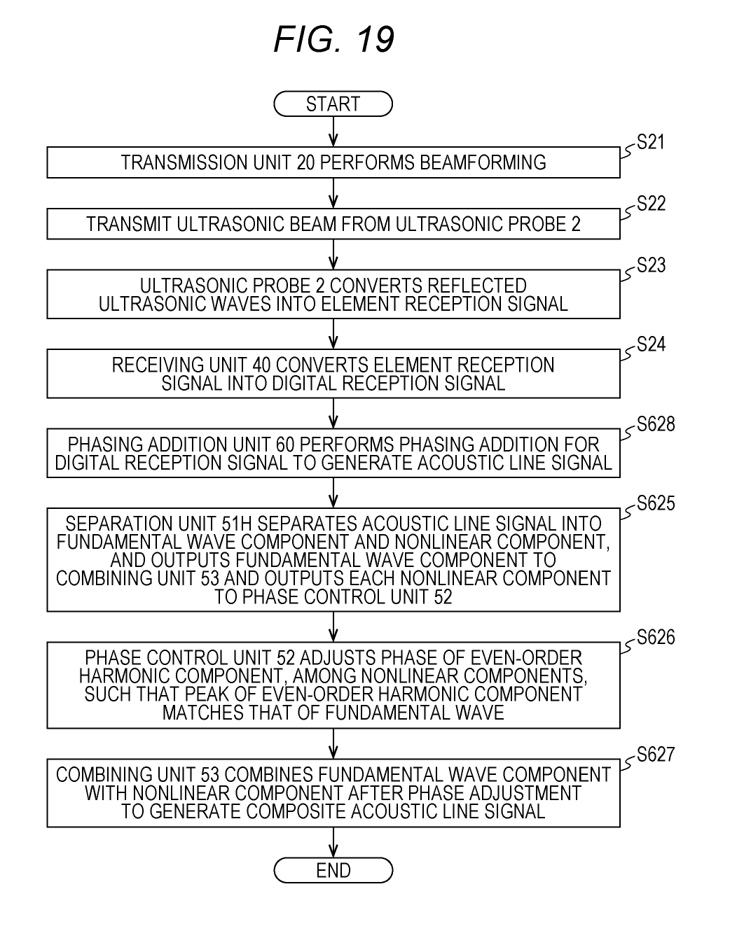

The operation of the ultrasonic diagnostic apparatus 7 according to the fifth embodiment will be described. The operation of ultrasonic diagnostic apparatus 7 is characterized in that the contents of the transmission and reception event are different, and operations other than the transmission and reception event are the same as those of the ultrasonic diagnostic apparatus 1. Hereinafter, the transmission and reception event will be described. FIG. 19 is a flowchart showing the operation of the transmission and reception event in the ultrasonic diagnostic apparatus 7. In addition, the same operations as in FIG. 3 are denoted by the same step numbers, and the detailed explanation thereof will be omitted.

First, the transmission unit 20 performs transmission beamforming (step S21).

Then, an ultrasonic beam is transmitted to the inside of the subject from the ultrasonic probe 2 (step S22).

Then, the reflected ultrasonic waves obtained from the inside of the subject by the ultrasonic probe 2 are converted into an element reception signal (step S23).

Then, the receiving unit 40 converts each element reception signal into a digital reception signal (step S24).

Then, the phasing addition unit 60 performs phasing addition for the digital reception signal to generate an acoustic line signal (step S628).

Then, the separation unit 51H separates the acoustic line signal into the fundamental wave component and a nonlinear component (step S625).

Then, the phase control unit 52H performs phase control for the nonlinear component after estimation correction (step S626).

Finally, the combining unit 53H combines the nonlinear component after the phase control with the fundamental wave to generate a composite acoustic line signal (step S627). In addition, although separation into respective components and phase control are the same as those in the first embodiment herein, the configurations of the first to third modification examples may be applied. For example, separation into respective components may be performed using the phase inversion method, and odd-order harmonic components may be directly output to the combining unit 53H. In addition, only the fundamental wave component and the odd harmonics group or both of the even harmonics group and the fundamental wave component and the odd harmonics group may be subjected to phase control processing.

In addition, pulse compression using correlation processing may be performed for the composite acoustic line signal or each component after phase control (after separation for a component that is not a phase control target) by applying the second or third embodiment.

In addition, estimation reproduction of the nonlinear component may be performed by applying the fourth embodiment.

<Summary>

Through the configuration described above, separation of the fundamental wave component and the nonlinear component, phase adjustment of the nonlinear component, and combination of the fundamental wave component and the nonlinear component after phase adjustment can be performed for each acoustic line signal instead of each digital reception signal. Therefore, it is possible to reduce the amount of computation.

### Other Modification Examples According to the Embodiments

(1) In each of the above embodiments and modification examples, the case of performing focus type beamforming in the transmitted ultrasonic beam has been described. However, for example, a transmitted ultrasonic beam may be transmitted as a plane wave, and an acoustic line signal of the entire region of interest may be generated for one transmission. In this case, it is possible to improve the frame rate of a B-mode image by reducing the number of times of the transmission and reception event required to generate the data of one B-mode image. In addition, in transmission beamforming and reception beamforming are not limited to the case described above, and any beamforming, such as a composite aperture method, may be used.

(2) In the second modification example, the case of using two fundamental wave components having different frequency bands has been described. However, for example, three or more fundamental wave components having different frequencies may be used.

In addition, in the second to fourth embodiments, two or more fundamental wave components may be used as in the second modification example. For example, pulse compression of the difference frequency or the sum frequency may be performed by correlation processing, or estimation reproduction may be performed.

(3) In each of the above embodiments and modification examples, the ultrasonic diagnostic apparatus generates one B-mode image. However, for example, the ultrasonic diagnostic apparatus may generate a plurality of B-mode images consecutively, and the plurality of B-mode images may be displayed on a display unit as a moving image. In addition, the generated B-mode image may be output to a storage medium or other devices, or the acoustic line signal may be output to a storage medium or other devices.

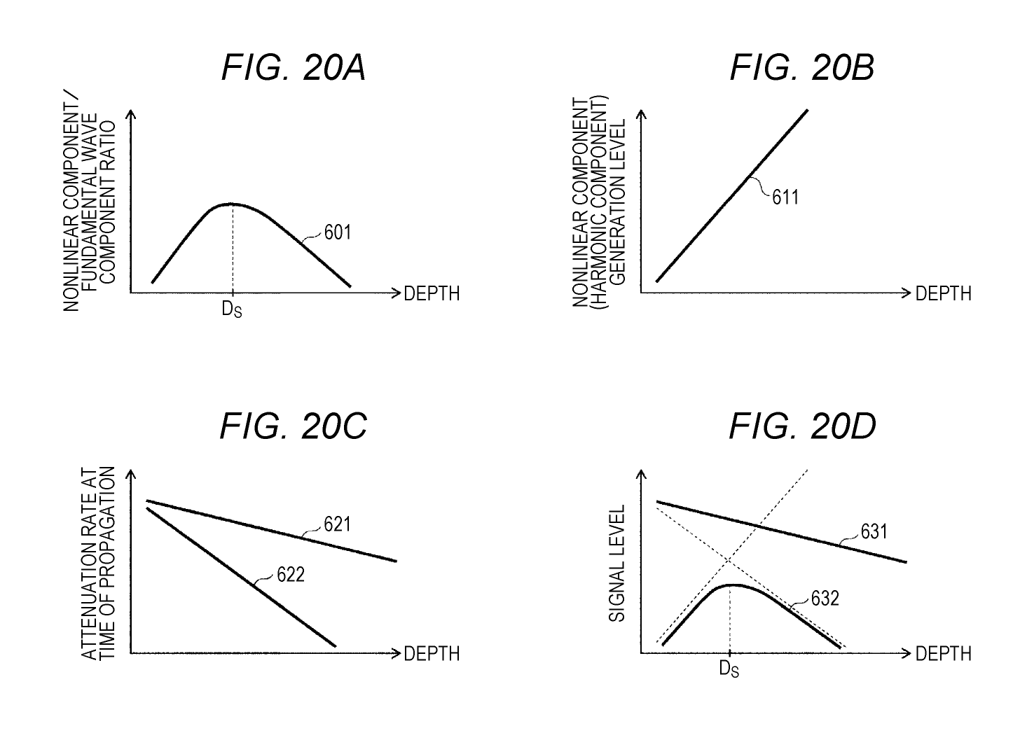

(4) In each of the above first, second, and fourth embodiments and modification examples, the combining unit combines the fundamental wave component and the nonlinear component in a composite predetermined ratio. However, the combination ratio of the nonlinear component and the fundamental wave component is not always fixed. For example, the percentage of the nonlinear component may be changed according to the conditions. Through the configuration, it is possible to further enhance the effect of pulse steepening. In this case, the combination ratio of the nonlinear component and the fundamental wave component may be simply set such that the percentage of the nonlinear component increases as the depth increases. Through the configuration, it is possible to obtain the effect of pulse steepening at any depth. Alternatively, for example, the combination ratio may be set such that the percentage of a component having a higher frequency is higher. Through the configuration, it is possible to enhance the effect of pulse steepening. Alternatively, for example, a combination ratio 601 shown in FIG. 20A may be used. In the combination ratio 601, the percentage of the nonlinear component is high when the depth is a predetermined depth Ds, and the percentage of the fundamental wave component is high when the depth is smaller than the predetermined depth Ds or when the depth is larger than the predetermined depth Ds. This is based on the following reasons. FIG. 20B shows the relationship between the generation level of the nonlinear component and the depth. Nonlinear components are generated by the propagation of ultrasonic waves. Therefore, as shown by a relationship 611, the generation level of the nonlinear component increases as the depth increases. On the other hand, FIG. 20C shows the relationship between the depth and the attenuation rate at the time of propagation. In general, attenuation due to propagation becomes larger as the frequency becomes higher. The nonlinear component has a higher frequency than the fundamental wave component. Accordingly, assuming that the relationship between the depth and the attenuation rate in the fundamental wave component is shown in a relationship 621, the nonlinear component is attenuated more largely as the depth increases as shown in a relationship 622. Due to these two factors, the relationship between the signal level of each of the fundamental wave component and the nonlinear component and the depth becomes a relationship shown in FIG. 20D. In FIG. 20D, a relationship 631 shows a relationship between the signal level of the fundamental wave component and the depth, and a relationship 632 shows a relationship between the signal level of the nonlinear component and the depth. A fundamental wave component is generated by the reflection of the fundamental wave component of the ultrasonic wave transmitted from the ultrasonic probe 2. Accordingly, since the fundamental wave component is not generated by propagation, it is sufficient to consider only the attenuation due to the propagation. That is, the signal level of the fundamental wave component decreases as the depth of the reflection point simply increases. On the other hand, the nonlinear component shows the following tendencies. In a shallow portion, since the generation level itself of the nonlinear component is low even though the attenuation rate is low, the signal level of the nonlinear component decreases as the depth decreases. On the other hand, in a deep portion, since the attenuation rate is high even though the generation level of the nonlinear component is high, the signal level of the nonlinear component decreases as the depth increases. Contrary to these, in the vicinity of the depth Ds, the signal level of the nonlinear component is not too low and the attenuation rate is not too high. Accordingly, the signal level of the nonlinear component is maximized. Eventually, the signal level of the nonlinear component increases as the depth approaches the depth Ds and decreases as the depth becomes away from the depth Ds. Therefore, the combination ratio of the fundamental wave component and the nonlinear component is set such that the percentage of the nonlinear component is high in a case where the signal level of the nonlinear component is high and the percentage of the fundamental wave component is high in a case where the signal level of the nonlinear component is low. This is because the effect of pulse narrowing is further enhanced if the percentage of the nonlinear component is increased in a case where the signal level of the nonlinear component is high while signal quality degradation due to noise mixing may become noticeable if the percentage of the nonlinear component is increased in a case where the signal level of the nonlinear component is low. Therefore, it is preferable to increase the percentage of the nonlinear component in the vicinity of the predetermined depth Ds and to decrease the percentage of the nonlinear component as the depth becomes away from the predetermined depth Ds, and it is possible to use the combination ratio 601 shown in FIG. 20A. In addition, the combination ratio is not limited to the combination ratio 601 shown in FIG. 20A. For example, when the depth is in the vicinity of the predetermined depth Ds, the percentage of the nonlinear component with respect to the fundamental wave component may be set to x. When the depth is not in the vicinity of the predetermined depth Ds, the percentage of the nonlinear component with respect to the fundamental wave component may be set to y (x>y). In addition, y may be zero (y=0).

In addition, although the combining unit combines time-series component signals in the third embodiment, a weighting may be similarly given to each of the time-series component signals, for example. In this case, the combination ratio of the fundamental wave component and the nonlinear component described above can be applied to the weighting coefficient of each of the time-series component signal obtained by compressing the fundamental wave component and the time-series component signal obtained by compressing the nonlinear component.

In addition, in the explanation of FIGS. 20A to 20D, the combination ratio of the fundamental wave component and the nonlinear component is changed according to the depth. However, the conditions for changing the combination ratio are not limited only to the depth, and changing the combination ratio may be changed according to a diagnostic part or other factors.

(5) The case of performing pulse compression using correlation processing for the composite reception signal has been described in the second embodiment, and the case of performing pulse compression using correlation processing for the fundamental wave component and each nonlinear component has been described in the third embodiment. However, for example, the pulse compression using correlation processing may be performed for each component of the even harmonics group after phase control. On the other hand, for the fundamental wave component and the odd-order harmonic component, the pulse compression using correlation processing may be performed in a state in which the fundamental wave component and the odd-order harmonic component are combined. Then, the results may be combined. On the contrary, the pulse compression using correlation processing may be performed for the fundamental wave component and each component of the odd-order harmonic component after phase control. On the other hand, for the even-order harmonic component, the pulse compression using correlation processing may be performed in a state in which the even-order harmonic components are combined. Then, the results may be combined. Alternatively, components of the even harmonics group may be combined after phase control, and the pulse compression using correlation processing may be performed for the entire even harmonics group after combination. On the other hand, for the fundamental wave component and the odd harmonics group, the pulse compression using correlation processing may be performed in a state in which the fundamental wave component and each component of the odd harmonics group are combined. Then, the results may be combined (needless to say, the even harmonics group may be replaced with the fundamental wave component and the odd harmonics group, and the fundamental wave component and the odd harmonics group may be replaced with the even harmonics group).

(6) In each of the above embodiments and modification examples, the ultrasonic probe 2 includes a plurality of transducers arranged in the one-dimensional direction. However, for example, the ultrasonic probe 2 may be a convex type probe, or transducers may be arranged in a two-dimensional direction. In addition, the ultrasonic probe 2 may include all or some of the switching unit 30, the transmission unit 20, and the receiving unit 40. In addition, although the ultrasonic probe 2 and the display unit 3 are configured so as to be connectable to the ultrasonic diagnostic apparatus, the ultrasonic probe 2 and the display unit 3 may be built into the ultrasonic diagnostic apparatus.

(7) In each of the above embodiments and modification examples, an example of the configuration is shown. However, the embodiments and the modification examples may be combined freely. For example, in the second modification example or the second to fourth embodiments, the separation unit 51 may separate the fundamental wave component and the nonlinear component from each other using the phase inversion method as in the first modification example. In addition, in the second to fourth embodiments, as in the second modification example, one or both of the difference frequency and the sum frequency may be performed in the same manner as for the nonlinear component using two or more fundamental waves having different frequencies. In addition, the fourth embodiment and the second or third embodiment may be combined, so that pulse compression may be performed for the nonlinear component estimated and reproduced by the estimation unit 100 or the composite reception signal including the nonlinear component.

(8) In the ultrasonic diagnostic apparatus according to each of the above embodiments and modification examples, all or some of the components may be implemented as one chip or an integrated circuit of a plurality of chips, or may be implemented as a computer program, or may be implemented in any other forms. For example, the separation unit, the phase control unit, and the combining unit may be implemented as one chip, or only the transmission signal generation unit may be implemented as one chip and an ultrasonic transducer unit and the like may be implemented as another chip.

In the case of implementing the components in an integrated circuit, the components are typically implemented as a large scale integration (LSI). Although the integrated circuit is an LSI herein, the integrated circuit may be called an IC, a system LSI, a super LSI, and an ultra LSI depending on the degree of integration.

In addition, the method of circuit integration may be realized using a dedicated circuit or a general-purpose processor without being limited to the LSI. After LSI manufacture, a field programmable gate array (FPGA) that can be programmed or a reconfigurable processor that can reconfigure the connections or settings of circuit cells in the LSI may be used.

In addition, if integrated circuit technology that replaces the LSI appears with the progress of semiconductor technology or other technologies, it is needless to say that the functional blocks may be integrated using the technology.

In addition, the ultrasonic diagnostic apparatus according to each of the above embodiments and modification examples may be implemented by a program written in a storage medium and a computer that reads and executes the program. The storage medium may be any recording medium, such as a memory card and a CD-ROM. In addition, the ultrasonic diagnostic apparatus according to the invention may be implemented by a program downloaded through a network or by a computer that downloads a program through a network and executes the program.

(9) The embodiments described above show preferable examples of the invention. Numeric values, shapes, materials, components, and arrangement positions and connected forms of components, steps, the order of steps, and the like described in the embodiments are just examples, and are not intended to limit the invention. In addition, among the components in the embodiments, a step that is not described in the independent claim and indicates the topmost concept of the invention is described as an optional component that forms a more preferable embodiment.

In addition, for easy understanding of the invention, reduced scales of the components in the diagrams mentioned in the above embodiments may be different from actual ones. In addition, the invention is not limited by the description of the above embodiments, and can be appropriately modified within the scope of the invention.

In addition, in the ultrasonic diagnostic apparatus, members, such as circuit components and lead wires, are also present on the substrate. For the electrical wires and electrical circuits, various forms can be implemented based on the ordinary knowledge in the art. Since these are not directly related to the description of the invention, the explanation has been omitted. In addition, each diagram shown above is a schematic diagram, and is not necessarily exactly shown.

<<Supplement>>

(1) An ultrasonic diagnostic apparatus according to an embodiment is an ultrasonic diagnostic apparatus that transmits and receives an ultrasonic wave to and from a subject using an ultrasonic probe and generates an image based on a reflected ultrasonic wave. The ultrasonic diagnostic apparatus includes: a transmission unit that converts a pulsed transmission signal including a fundamental wave component into a transmission ultrasonic wave using the ultrasonic probe and transmits the transmission ultrasonic wave to the inside of the subject; a receiving unit that generates a reception signal based on a reflected ultrasonic wave from the subject that has been received by the ultrasonic probe; a separation unit that separates the reception signal into a first component including one or more frequency components and a second component different from the first component; a phase control unit that generates a third component by controlling a phase of the second component such that a time at which amplitude is maximized is the same between the first and second components; a combining unit that combines the first and third components to generate a composite reception signal; and an image generation unit that generates an image based on the composite reception signal.

In addition, an ultrasonic signal processing method according to an embodiment includes: converting a pulsed transmission signal including a fundamental wave component into a transmission ultrasonic wave using an ultrasonic probe and transmitting the transmission ultrasonic wave to the inside of a subject; generating a reception signal based on a reflected ultrasonic wave from the subject that has been received by the ultrasonic probe; separating the reception signal into a first component including one or more frequency components and a second component different from the first component; generating a third component by controlling a phase of the second component such that a time at which amplitude is maximized is the same between the first and second components; combining the first and third components to generate a composite reception signal.

Through the configuration described above, since the first and second components are strengthened by interaction, the peak of the composite reception signal becomes steep. As a result, it is possible to improve the distance resolution by shortening the substantial pulse length. In addition, since the phases of the first and second components do not need to match each other in the initial state of the reception signal, a plurality of different frequency components included in the reception signal can be used as the first and second components. As a result, it is possible to widen the band of the signal.

(2) In addition, in the ultrasonic diagnostic apparatus described in (1) or the ultrasonic signal processing method, one of the first and second components may be a fourth component including a reflected fundamental wave component having the same frequency band as the fundamental wave component, and the other one may be a fifth component including even-order harmonic components of the reflected fundamental wave component.

Through the configuration described above, one of the reflected fundamental wave component and the even-order harmonic component, which is a nonlinear component, can be used as the first component, and the other one can be used as the second component.

(3) In addition, in the ultrasonic diagnostic apparatus described in (1) or (2) or the ultrasonic signal processing method, the transmission signal may include the fundamental wave component and a component having a frequency of M (M is an integer of 2 or more) times a frequency of the fundamental wave component.

Through the configuration described above, since the nonlinear component generated by the propagation of the fundamental wave component and the reflected wave of the component having a frequency of M times the frequency of the fundamental wave component can be made to strengthen each other, it is possible to improve the signal strength of the nonlinear component.

(4) In addition, in the ultrasonic diagnostic apparatus described in (2) or (3) or the ultrasonic signal processing method, the fourth component may further include odd-order harmonic components of the reflected fundamental wave component.

Through the configuration described above, the odd-order harmonic component that is a nonlinear component can be further used as the first component or the second component that includes the reflected fundamental wave.

(5) In addition, in the ultrasonic diagnostic apparatus described in any one of (2) to (4), the transmission signal may further include a second fundamental wave component having a different frequency from the fundamental wave component, and the fifth component may further include one or both of a sum frequency component between the fundamental wave component and the second fundamental wave component and a difference frequency component between the fundamental wave component and the second fundamental wave component.

Through the configuration described above, it is possible to generate a composite reception signal configured to include one of two fundamental wave components having different frequencies and a sum frequency component and/or a difference frequency component.

(6) In addition, in the ultrasonic diagnostic apparatus described in (5), the fourth component may further include one or both of a second reflected fundamental wave component corresponding to the second fundamental wave component and odd-order harmonic components of the second reflected fundamental wave component, and the fifth component may further include even-order harmonic components of the second reflected fundamental wave component.

Through the configuration described above, any one or more of the odd-order harmonic component and the reflected fundamental wave component corresponding to each of two fundamental wave components having different frequencies and any one or more of the sum frequency component, the difference frequency component, and the even-order harmonic component corresponding to each fundamental wave component can be used as one and the other of the first and second components, respectively.

(7) In addition, in the ultrasonic diagnostic apparatus described in any one of (1) to (6), the phase control unit may generate a sixth component by further controlling a phase of the first component such that a time at which amplitude is maximized is the same between the third and sixth components, and the combining unit may generate the composite reception signal using the sixth component instead of the first component.

Through the configuration described above, more suitable phase control can be performed by setting both the first and second components as phase control targets.

(8) In addition, the ultrasonic diagnostic apparatus described in anyone of (2) to (7) may further include an estimation unit that estimates and generates restored harmonic components, which are waveforms before degradation of harmonic components of the reflected fundamental wave component, using the reflected fundamental wave component. The phase control unit may generate the third component by controlling a phase of a seventh component obtained by replacing harmonic components of the reflected fundamental wave component of the second component with the restored harmonic components, and the combining unit may generate the composite reception signal using an eighth component, which is obtained by replacing harmonic components of the reflected fundamental wave component of the first component with the restored harmonic components, instead of the first component.

Through the configuration described above, since it is possible to increase the signal level of the harmonic component while maintaining the quality of the harmonic component, it is possible to make the peak of the composite reception signal steeper. As a result, it is possible to improve the distance resolution more reliably.

(9) In addition, in the ultrasonic diagnostic apparatus described in any one of (2) to (8), the combining unit may control a combination ratio between a ninth component corresponding to the reflected fundamental wave component and a tenth component corresponding to harmonic components of the reflected fundamental wave component when generating the composite reception signal.

Through the configuration described above, it is possible to use harmonic components more appropriately. As a result, it is possible to suppress the degradation of the signal quality and to improve the distance resolution by making the peak of the composite reception signal steeper.

(10) In addition, in the ultrasonic diagnostic apparatus described in (9), the combining unit may change the combination ratio of the tenth component to the ninth component according to a depth of a generation source of the reflected ultrasonic wave corresponding to the reception signal.

Through the configuration described above, it is possible to make the peak of the composite reception signal steep efficiently in consideration of the attenuation or the signal level of the harmonic component. As a result, it is possible to increase the distance resolution while maintaining the quality of the harmonic component.

(11) In addition, in the ultrasonic diagnostic apparatus described in (10), the combination ratio of the tenth component to the ninth component may increase as a depth of a generation source of the reflected ultrasonic wave corresponding to the reception signal increases when the depth of the generation source is smaller than a predetermined depth, and may decrease as the depth of the generation source increases when the depth of the generation source is larger than the predetermined depth.

Through the configuration described above, the effect of peak steepening of the composite reception signal can be enhanced by increasing the percentage of the harmonic component in the vicinity of the predetermined depth where the signal level of the harmonic component is high, while quality degradation of the composite reception signal due to noise included in the harmonic component can be suppressed by reducing the percentage of the harmonic component for a region away from the predetermined depth where the signal level of the harmonic component is low.

(12) In addition, the ultrasonic diagnostic apparatus described in any one of (1) to (11) may further include a pulse compression unit that generates a pulse compression signal by compressing the composite reception signal in a time axis direction based on the transmission signal, and the image generation unit may generate the image based on the pulse compression signal instead of the composite reception signal.

Through the configuration described above, since it is possible to make the peak of the composite reception signal steeper, it is possible to improve the distance resolution more reliably.

(13) In addition, the ultrasonic diagnostic apparatus described in any one of (1) to (6) may further include a pulse compression unit that generates a first pulse compression signal and a second pulse compression signal by compressing the first component and the third component in a time axis direction based on the transmission signal, respectively, and the combining unit may generate the composite reception signal by combining the first and second pulse compression signals instead of the first and third components.

Through the configuration described above, since it is possible to match the timings of the peaks of the first and second pulse compression signals, it is possible to improve the distance resolution more reliably.

(14) In addition, in the ultrasonic diagnostic apparatus described in any one of (1) to (12), the phase control unit may change a phase of each frequency component included in the second component by π/2.

Through the configuration described above, it is possible to reduce the amount of computation for phase control.

The ultrasonic diagnostic apparatus and the ultrasonic signal processing method according to an embodiment of the invention do not require a complicated circuit, and it is possible to improve the S/N ratio and the distance resolution using nonlinear components. In addition, in a region where it is not possible to receive nonlinear components, imaging based on the fundamental wave component is possible. Accordingly, there is a high adaptability that is not influenced by the conditions of use in a medical diagnostic apparatus or the like.

Although the present invention has been described and illustrated in detail, it is clearly understood that the same is by way of illustrated and example only and is not to be taken by way of limitation, the scope of the present invention being interpreted by terms of the appended claims.

What is claimed is:

1. An ultrasonic diagnostic apparatus that transmits and receives an ultrasonic wave to and from a subject using an ultrasonic probe and generates an image based on a reflected ultrasonic wave, the ultrasonic diagnostic apparatus comprising:
a transmission unit that converts a pulsed transmission signal including a fundamental wave component into a transmission ultrasonic wave using the ultrasonic probe and transmits the transmission ultrasonic wave to the inside of the subject;
a receiving unit that generates a reception signal based on a reflected ultrasonic wave from the subject that has been received by the ultrasonic probe;
a separation unit that separates the reception signal into a first component including one or more frequency components and a second component different from the first component;
a phase control unit that generates a third component by controlling a phase of the second component such that a time at which amplitude is maximized is the same between the first and second components;
a combining unit that combines the first and third components to generate a composite reception signal; and
an image generation unit that generates an image based on the composite reception signal.

2. The ultrasonic diagnostic apparatus according to claim 1,
wherein one of the first and second components is a fourth component including a reflected fundamental wave component having the same frequency band as the fundamental wave component, and the other one is a fifth component including even-order harmonic components of the reflected fundamental wave component.

3. The ultrasonic diagnostic apparatus according to claim 1,
wherein the transmission signal includes the fundamental wave component and a component having a frequency of M (M is an integer of 2 or more) times a frequency of the fundamental wave component.

4. The ultrasonic diagnostic apparatus according to claim 2,
wherein the fourth component further includes odd-order harmonic components of the reflected fundamental wave component.

5. The ultrasonic diagnostic apparatus according to claim 2,
wherein the transmission signal further includes a second fundamental wave component having a different frequency from the fundamental wave component, and
the fifth component further includes one or both of a sum frequency component between the fundamental wave component and the second fundamental wave component and a difference frequency component between the fundamental wave component and the second fundamental wave component.

6. The ultrasonic diagnostic apparatus according to claim 5,
wherein the fourth component further includes one or both of a second reflected fundamental wave component corresponding to the second fundamental wave component and odd-order harmonic components of the second reflected fundamental wave component, and
the fifth component further includes even-order harmonic components of the second reflected fundamental wave component.

7. The ultrasonic diagnostic apparatus according to claim 1,
wherein the phase control unit generates a sixth component by further controlling a phase of the first component such that a time at which amplitude is maximized is the same between the third and sixth components, and
the combining unit generates the composite reception signal using the sixth component instead of the first component.

8. The ultrasonic diagnostic apparatus according to claim 2, further comprising:
an estimation unit that estimates and generates restored harmonic components, which are waveforms before degradation of harmonic components of the reflected fundamental wave component, using the reflected fundamental wave component,
wherein the phase control unit generates the third component by controlling a phase of a seventh component obtained by replacing harmonic components of the reflected fundamental wave component of the second component with the restored harmonic components, and
the combining unit generates the composite reception signal using an eighth component, which is obtained by replacing harmonic components of the reflected fundamental wave component of the first component with the restored harmonic components, instead of the first component.

9. The ultrasonic diagnostic apparatus according to claim 2,
wherein the combining unit controls a combination ratio between a ninth component corresponding to the reflected fundamental wave component and a tenth component corresponding to harmonic components of the reflected fundamental wave component when generating the composite reception signal.

10. The ultrasonic diagnostic apparatus according to claim 9,
wherein the combining unit changes the combination ratio of the tenth component to the ninth component according to a depth of a generation source of the reflected ultrasonic wave corresponding to the reception signal.

11. The ultrasonic diagnostic apparatus according to claim 10,
wherein the combination ratio of the tenth component to the ninth component increases as a depth of a generation source of the reflected ultrasonic wave corresponding to the reception signal increases when the depth of the generation source is smaller than a predetermined depth, and decreases as the depth of the generation source increases when the depth of the generation source is larger than the predetermined depth.

12. The ultrasonic diagnostic apparatus according to claim 1, further comprising:
a pulse compression unit that generates a pulse compression signal by compressing the composite reception signal in a time axis direction based on the transmission signal,
wherein the image generation unit generates the image based on the pulse compression signal instead of the composite reception signal.

13. The ultrasonic diagnostic apparatus according to claim 1, further comprising:
a pulse compression unit that generates a first pulse compression signal and a second pulse compression signal by compressing the first component and the third component in a time axis direction based on the transmission signal, respectively,
wherein the combining unit generates the composite reception signal by combining the first and second pulse compression signals instead of the first and third components.

14. The ultrasonic diagnostic apparatus according to claim 1,
wherein the phase control unit changes a phase of each frequency component included in the second component by π/2.

15. An ultrasonic signal processing method, comprising:
converting a pulsed transmission signal including a fundamental wave component into a transmission ultrasonic wave using an ultrasonic probe and transmitting the transmission ultrasonic wave to the inside of a subject;
generating a reception signal based on a reflected ultrasonic wave from the subject that has been received by the ultrasonic probe;
separating the reception signal into a first component including one or more frequency components and a second component different from the first component;
generating a third component by controlling a phase of the second component such that a time at which amplitude is maximized is the same between the first and second components; and
combining the first and third components to generate a composite reception signal.

16. The ultrasonic signal processing method according to claim 15,
wherein one of the first and second components is a fourth component including a reflected fundamental wave component having the same frequency band as the fundamental wave component, and the other one is a fifth component including even-order harmonic components of the reflected fundamental wave component.

17. The ultrasonic signal processing method according to claim 16,
wherein the fourth component further includes odd-order harmonic components of the fundamental wave component.
